(19) United States
(12) Patent Application Publication
(10) Pub. No.: US 2025/0341511 A1
(43) Pub. Date: Nov. 6, 2025

(54) MASS-TAG LABELING OF THE CELLULAR SECRETOME

(71) Applicants: The Governing Council of the University of Toronto, Toronto (CA); Universität zu Köln, Köln (DE)

(72) Inventors: Daniel Bachurski, Koln (DE); Michael Hallek, Koln (DE); Mark Nitz, Toronto (CA); Yong Jia Bu, Toronto (CA)

(21) Appl. No.: 18/862,127

(22) PCT Filed: May 5, 2023
(86) PCT No.: PCT/EP2023/062049

§ 371 Date: Oct. 31, 2024

Related U.S. Application Data
(60) Provisional application No. 63/339,897, filed on May 9, 2022.
(60) Provisional application No. 63/345,781, filed on May 25, 2022.

(51) Int. Cl. G01N 33/50

(52) U.S. Cl. CPC G01N 33/5035

(57) ABSTRACT

The present disclosure provides methods for mass-tag labeling of the cellular secretome and soluble components thereof, as well as mass-tagged soluble components of the cellular secretome. In certain embodiments, the disclosure provides methods for mass-tagging of extracellular vesicles (EVs) and mass-tagged EVs. Also provided are methods of using mass-tagged soluble components of the cellular secretome, such as mass-tagged EVs, mass-tagged viruses, or mass-tagged soluble proteins and peptides. These can be combined with other labeling strategies, such as cell barcoding to facilitate multiplexed and/or multi-dimensional analyses of the distribution, uptake, and effects of components of secretome (such as EVs).

### CROSS-REFERENCE TO RELATED APPLICATIONS

This application claims benefit of and priority to U.S. Ser. No. 63/339,897, filed on May 9, 2022, and U.S. Ser. No. 63/345,781, filed on May 25, 2022 which are incorporated herein by reference in their entirety for all purposes.

### FIELD OF THE DISCLOSURE

The present disclosure relates to generally to the area of mass-tag labeling soluble, cell derived components with elemental isotopic compositions. In particular, the disclosure relates to labeling components of the cellular secretome, including extracellular vesicles (EVs) and related compositions and methods.

### BACKGROUND OF THE DISCLOSURE

The cellular secretome encompasses a collection of soluble proteins, extracellular vesicles, and other biomolecules secreted by cells into the extracellular environment. These molecules play a critical role in cell-cell communication, tissue development, and disease progression. In recent years, there has been growing interest in studying the cellular secretome to better understand intercellular communication and extracellular functions and to identify potential therapeutic targets.

Extracellular vesicles (EVs) are membrane-enclosed biological nanoparticles secreted by virtually every known cell type [1]. As key actors in intercellular communication and essential components of the cellular secretome, EVs are increasingly investigated as therapeutic agents in several disease entities [2,3]. Moreover, EVs can carry several layers of information, such as RNA species, extra and intravesicular proteins, and lipids [4]. However, their exact biological function, target or recipient cells, and biodistribution after in vivo application remain largely unknown [5]. Due to the physical (small size, low refractive index) and biochemical (weak expression of proteins and RNAs) properties of EVs [6], the examination of the unknowns mentioned above is predominantly dependent on EV labeling strategies [7].

Mass cytometry and imaging mass cytometry have emerged as powerful methods for analyzing cellular phenotypes within heterogeneous cell populations. approaches involve the antibody-based and mass tag-dependent identification and quantification of protein targets in complex biological samples using mass spectrometric analyses of heavy metal isotopes. Mass spectrometry-based single-cell techniques, such as cytometry by Time-Of-Flight (CyTOF) or imaging mass cytometry, have been widely used for cellular and tissue analysis but have not yet been employed for recipient cell analysis of various components of the cellular secretome.

Several mass-tagging techniques have been developed to label whole cells or measure their diverse intracellular functions for (imaging) mass cytometric analyses. Mass-tag labeling has been primarily employed to examine the phenotype or cellular function of target cells.

### SUMMARY OF THE DISCLOSURE

To study the interactions and effects of cellular secretome components, such as EVs) on recipient cells' phenotype and function, a mass-tagging approach requires uniform and normalized labeling of various secretome components, including extracellular vesicles (EVs), soluble proteins (e.g., antibodies, hormones, cytokines, and enzymes), and viruses. A prerequisite for one approach to this uniform labeling is the metabolic labeling of secretome-producing cells.

With respect to EVs, in most cases, fluorescent lipophilic dyes, protein-labeling probes, or genetic labeling approaches are applied to analyze the impact of EV uptake on cellular function and signaling in several in vivo and in vitro models [7]. Both tagging strategies are unsuitable for high-dimensional single-cell analyses of recipient cells in vitro and in vivo and show additional extensive limitations rendering the study of the functional impact of EVs on their recipient cells complicated [8]: Lipophilic dyes and fluorescent probes are often self-aggregating, leading to the artificial formation of EV-like structures that are taken up by cells and tissues causing false-positive signals [9]. Genetic labeling strategies are mostly limited to single proteins not present in all secreted EV subpopulations (CD9, CD63, or CD81, e.g.) and cannot be used with most primary cell types [7]. Additionally, fluorescent genetic tags are large and might sterically hinder protein-protein interactions and thus EV uptake and function. Other labeling strategies are desirable, especially for use in high-dimensional single-cell analysis.

Cellular components can be labeled by mass tagging. However, it has not been demonstrated previously that mass-tagged components of the cellular secretome can be actively secreted after being mass-tag labeled within the cell and subsequently detected in the extracellular space and in recipient cells. The secretion process could potentially be hindered by the integration of the mass tag, which might result in alterations to the protein's binding and phenotype. Moreover, it remained unknown according to the same reason whether mass-tagged components of the cellular secretome could be internalized by recipient cells—an additional requirement for conducting cellular secretome recipient cell analyses by mass cytometry and respective imaging techniques.

We aimed to overcome most limitations mentioned above by creating a cellular secretome, e.g., an EV, mass-tag labeling approach for traceability with high-dimensional single-cell mass cytometry and mass cytometry imaging, in the case of EVs, following the MISEV2018 EV criteria [10]. We demonstrated that EVs can be mass-tagged without substantially altering MISEV2018 characteristics. We found our mass-tag labeling also suitable for labeling all parts of the cellular secretome, including EVs, secreted soluble proteins (such as antibodies, cytokines, hormones, e.g.), viruses, and intracellular pathogens.

Although direct labeling of extracellular vesicle subpopulations has been performed based on nucleic acid intercalation (e.g., Intercalator-Ir/Rh, IdU) or alkylation (e.g., DDP, Cisplatin), the reported labeling approaches have significant disadvantages that the proteomic and metabolic mass-tag labeling approach of the cellular secretome circumvents, including:

Existing labeling approaches for extracellular vesicles (EVs) are not suitable for simultaneously labeling various components of the cellular secretome and only label a fraction of a single component of the cellular secretome -DNA+EVs-. In contrast, our mass-tag labeling approach labels various components of the cellular secretome, including EVs and soluble proteins, uniformly and simultaneously, enabling labeling of all EV subpopulations as opposed to DNA-based labeling methods.

Direct labeling methods previously reported require EVs to be permeabilized by electroporation, which is time-consuming, expensive, and impractical, affecting morphology and causing EV aggregation. Our mass-tag labeling approach for the cellular secretome does not require electroporation or any other EV modification prior to the metabolic labeling within the producing cell.

The analysis of single components of the secretome is often biased by the presence of contaminants from other components of the cellular secretome. Biological effects (e.g., of antibodies or biologically active peptides) cannot be compared between several components of the cellular secretome when only single components of the cellular secretome are labeled. By applying our mass-tag labeling approach to the cellular secretome, proteins within the secretome are labeled uniformly and normalized based on the Gaussian distribution of mass-tag integration within the producing cell.

The novel mass-tag labeling of the cellular secretome described herein addresses the limitations of existing methods, as outlined above. This new approach offers the following advantages:

Improved labeling efficiency: Our method ensures more complete labeling of secreted peptides and proteins, resulting in more accurate quantification and comparison.

Enhanced multiplexing capability: Our approach allows for the simultaneous analysis of a larger number of samples, increasing throughput.

Simplified workflow and reduced cost: The proposed method streamlines the labeling process and reduces the associated cost.

Compatibility with mass cytometry, imaging mass cytometry, electron microscopy-based techniques, and other mass spectrometry-based single-cell and imaging techniques: Our method is specifically designed to work seamlessly with mass-spectrometry-based single-cell and imaging techniques and with electron microscopy offering a tailored solution for recipient cells of and mass-tag labeled components of the cellular secretome.

Adaptability: The novel mass-tag labeling approach can be easily adapted for various experimental setups and sample types, making it a versatile option for researchers, in contrast to electroporation-based techniques to label EVs.

Enhanced sensitivity and specificity: Our method provides increased sensitivity and specificity, allowing for the detection of low-protein-containing components, such as EVs and secreted soluble proteins at the same time.

Various embodiments contemplated herein may include, but need not be limited to, one or more of the following:

Embodiment 1: An extracellular vesicle (EV), wherein a component of the EV is labeled with at least one mass tag.

Embodiment 2: A plurality of EVs according to embodiment 1.

Embodiment 3: The plurality of EVs of embodiment 2, wherein the plurality comprises EVs from more than one sample.

Embodiment 4: The plurality of EVs of embodiment 3, wherein the EVs from each different sample are distinguished by a different detectable label or combination of detectable labels.

Embodiment 5: The plurality of EVs of embodiment 4, wherein the different labels or combinations of labels comprise different mass tags or combinations of mass tags.

Embodiment 6: A method of producing a mass-tagged soluble component from a production cell, the method comprising: exposing at least one production cell to a mass-tagged component that can be taken up by the production cell; and purifying a mass-tagged soluble component produced by the production cell.

Embodiment 7: The method of embodiment 6, wherein the mass-tagged soluble component is selected from an extracellular vesicle (EV), a virus particle, a cellular secretome, an EV proteome or secretome, or a component of any of the foregoing.

Embodiment 8: The method of embodiment 7, wherein the mass-tagged component is a mass-tagged EV.

Embodiment 9: The method of any one of embodiments 6-8, wherein the production cell is exposed to the mass-tagged component under serum-free conditions.

Embodiment 10: The method of any one of embodiments 6-9, wherein the production cell is derived from a cell line, optionally selected from HEK293T, HeLa, OSU-CLL, and PANC-1.

Embodiment 11: The method of embodiment 7 or embodiment 9, wherein the production cell is derived from a primary cell, optionally a chronic lymphocytic leukemia cell.

Embodiment 12: The method of any one of embodiments 7-11, wherein the EVs are purified by a method comprising filtration, ultrafiltration, and size-exclusion chromatography.

Embodiment 13: The method of embodiment 12, wherein the filtration comprises 0.2 μM filtration, the ultrafiltration comprises 10 kDa ultrafiltration, and the size-exclusion chromatograph comprises qEV/35 nm chromatography.

Embodiment 14: A method of producing a mass-tagged EV, the method comprising contacting the EV with a mass tag that is functionalized to bind to a component of the EV under conditions suitable for that binding to occur.

Embodiment 15: The method of embodiment 14, wherein the method additionally comprises purifying the EV from a bodily fluid or tissue before contacting the EV with the functionalized mass tag.

Embodiment 16: An EV produced according to the method of any one of embodiments 7-14.

Embodiment 17: An extracellular vesicle proteome or secretome from the EV of embodiment 16, wherein the proteome or secretome comprises a mass-tagged component.

Embodiment 18: A method of using the EV of embodiment 1, the method comprising: contacting the EV with a recipient cell, whereby the recipient cell takes up the EV.

Embodiment 19: The method of embodiment 18, wherein the method is an in vivo method, and the EV is used for diagnosis or therapy.

Embodiment 20: The method of embodiment 18, wherein the EV is used in a non-diagnostic and non-therapeutic method.

Embodiment 21: The method of embodiment 18, wherein the method is an in vitro method.

Embodiment 22: The method of embodiment 18, wherein the method comprises a biodistribution study.

Embodiment 23: The method of embodiment 19, wherein the method comprises analyzing a single recipient cell.

Embodiment 24: The method of embodiment 19, wherein the method comprises analyzing a plurality of recipient cells.

Embodiment 25: The method of embodiment 24, wherein the plurality of recipient cells comprises cells of different cell types.

Embodiment 26: The method of embodiment 18, wherein the method additionally comprises measuring a change in cellular function after EV uptake, as compared to before EV uptake, wherein the change in cellular function is optionally selected from apoptosis, DNA-damage response, migration, proliferation, and tyrosine-kinase signaling.

Embodiment 27: The method of any one of embodiments 18-26, wherein the recipient cell is labeled with at least one detectable label.

Embodiment 28: The method of embodiment 27, wherein the detectable label indicates a characteristic of the recipient cell.

Embodiment 29: The method of embodiment 28, wherein the characteristic of the recipient cell, alone or in combination with other characteristics, distinguishes the recipient cell type from at least one other cell type.

Embodiment 30: The method of embodiment 29, wherein the characteristic of the recipient cell, alone or in combination with other characteristics, identifies the recipient cell type.

Embodiment 31: The method of any one of embodiments 27-30, wherein the detectable label comprises a mass tag.

Embodiment 32: The method of any one of embodiments 27-30, wherein the recipient cell is subjected to CD45-based live cell barcoding or palladium-based fixed cell barcoding.

Embodiment 33: The method of embodiment 32, where the barcoding identifies cells from different samples and/or cells of different cell types.

Embodiment 34: The method of any one of embodiments 18-33, wherein the method comprises employing the detectably labeled recipient cell and/or one or more detectably labeled reagents to characterize EV uptake and/or EV-mediated effects, to identify recipient cells, and/or in a multiplex analysis, optionally wherein the one or more detectably labeled reagents are one or more antibodies.

Embodiment 35: The method of embodiment 34, wherein the detectably labeled recipient cells are labeled using a metal-labeled antibody panel and/or the one or more detectably labeled reagents comprise a metal-labeled antibody panel.

Embodiment 36: The method of any one of embodiments 18-31, wherein the method comprises performing a technique selected from mass cytometry, mass cytometry imaging, and transmission electron microscopy, or another mass spectrometry-based single-cell and/or imaging technique on the recipient cell.

Embodiment 37: A recipient cell produced by the method of embodiment 18.

Embodiment 38: A method of detecting the EV of embodiment 1 or embodiment 16 and or the recipient cell of embodiment 37, wherein the method comprises performing a technique selected from mass cytometry, mass cytometry imaging, and transmission electron microscopy, or another mass spectrometry-based single-cell and/or imaging technique.

Embodiment 39: A kit for performing the method of embodiment 6, wherein the kit comprises one or more mass-tagged components that can be taken up by a production cell.

Embodiment 40: The EV of embodiment 1 or embodiment 16, the plurality of EVs of any one of embodiments 2-5, the method of any one of embodiments 7-13, 18-36, or 38, the recipient cell of embodiment 37, or the kit of embodiment 39, wherein said mass-tagged component comprises an amino acid or analog thereof.

Embodiment 41: The EV, method, or kit of embodiment 40, wherein the amino acid is phenylalanine or an analog thereof.

Embodiment 42: The EV, method, or kit of embodiment 40 or embodiment 41, wherein a protein component of the EV, virus particle, or cellular or EV proteome or secretome is labeled with the at least one mass tag.

Embodiment 43: The EV of embodiment 1 or embodiment 16, the plurality of EVs of any one of embodiments 2-5, the method of any one of embodiments 7-13, 18-36, or 38, the recipient cell of embodiment 37, the EV or method of any one of embodiments 40-42, or the kit of embodiment 39, wherein the mass tag comprises an organotellurophene tag.

Embodiment 44: The EV, method, or kit of embodiment 43, wherein the organotellurophene tag comprises L-2-tellurienylalanine (TePhe) or TeMal.

Embodiment 45: The EV, method, or kit of embodiment 44, wherein a plurality of mass tags selected from isotopologues of TePhe or TeMal is provided or employed to facilitate multiplex analysis.

Embodiment 46: The EV or method of embodiment 43 or embodiment 44, wherein the mass-tagged EV does not differ substantially from an unlabeled EV produced from the same cell type under the same conditions as the labeled EV.

Embodiment 47: The EV or method of embodiment 46, wherein the mass-tagged EV and the unlabeled EV have substantially the same effect(s) on a recipient cell.

Embodiment 48: The EV or method of embodiment 47, wherein the effect(s) of the mass-tagged EV and the unlabeled EV differ by no more than ±15, ±14, ±13 ±12, ±11, ±10, ±9, ±8, ±7, ±6, ±5, ±4, ±3, ±2, ±1, 0.5% percent.

Embodiment 49: The EV or method of embodiment 46, wherein the mass-tagged EV and the unlabeled EV have substantially the same MISEV2018 characteristic(s) for one or more or all MISEV2018 characteristics.

Embodiment 50: The EV or method of embodiment 47, wherein the characteristic(s) of the mass-tagged EV and the unlabeled EV differ by no more than ±15, ±14, ±13 ±12, ±11, ±10, ±9, ±8, ±7, ±6, ±5, ±4, ±3, ±2, ±1, ±0.5% percent.

Embodiment 51: The use of a mass tag, characterized in that the mass tag is used to label the cellular secretome, and a mass-tagged component of the cellular secretome is purified.

Embodiment 52: The use of embodiment 51, characterized in that the cellular secretome is labeled by metabolic labeling.

Embodiment 53: The use of embodiment 51 or embodiment 52, characterized in that the mass-tagged component of the cellular secretome comprises one or a plurality of EV(s).

Embodiment 54: The use of any one of embodiments 51-53, characterized in that the mass-tagged component of the cellular component is used in a study with one or a plurality of other detectably labeled component(s).

Embodiment 55: The use of embodiment 54, characterized in that the study comprises a multiplex analysis.

Embodiment 56: A method for mass-tag labeling of the cellular secretome, comprising metabolic labeling of secretome-producing cells, including extracellular vesicles, soluble proteins and peptides, and viruses, with mass-tagged amino acids or analogs to simultaneously and uniformly label various components of the cellular secretome.

Embodiment 57: The method of embodiment 56, wherein the mass-tag labeling is compatible with mass cytometry, imaging mass cytometry, electron microscopy, and other mass spectrometry-based single-cell and imaging techniques.

Embodiment 58: The method of embodiment 56, further comprising the analysis of labeled cellular secretome components by mass cytometry, imaging mass cytometry, electron microscopy, or other mass spectrometry-based single-cell and imaging techniques.

Embodiment 59: The method of embodiment 56, wherein the mass-tag labeling does not require electroporation or any other modification of extracellular vesicles prior to metabolic labeling within the producing cell.

Embodiment 60: The method of embodiment 56, wherein the mass-tag labeling provides increased sensitivity and specificity for the detection of low-protein-containing components, such as extracellular vesicles and viruses.

Embodiment 61: A mass-tag labeled cellular secretome, extracellular vesicle, soluble protein, or virus produced by the method of embodiment 56.

Embodiment 62: The mass-tag labeled cellular secretome of embodiment 61, for use in biomarker discovery for disease diagnosis, prognosis, treatment response monitoring, drug target identification, biodistribution analysis, and basic research.

Embodiment 63: A kit for mass-tag labeling of the cellular secretome, comprising reagents for metabolic labeling of secretome-producing cells, including mass-tagged amino acids or analogs, and instructions for the mass-tag labeling of the cellular secretome and subsequent analysis of labeled cellular secretome components.

Embodiment 64: A method for identifying biomarkers, drug targets, or studying intercellular communication using the mass-tag labeled cellular secretome of embodiment 61.

Embodiment 65: The mass-tag labeled cellular secretome of embodiment 61, for use in proteomics research to quantify protein abundance, assess protein-protein interactions, and investigate post-translational modifications.

Embodiment 66: A mass-tag labeled cellular secretome recipient cell analysis system, comprising a mass cytometer, an imaging mass cytometer, an electron microscope, or other mass spectrometry-based single-cell and imaging instruments, and a mass-tag labeled cellular secretome prepared according to the method of embodiment 56.

Embodiment 67: The system of embodiment 66, further comprising a software package for the analysis and visualization of mass-tag labeled cellular secretome recipient cell data.

Embodiment 68: A method for normalizing and comparing mass-tag labeled components of the cellular secretome, wherein proteins within the secretome are labeled uniformly and normalized based on the Gaussian distribution of mass-tag integration, enabling more accurate quantification and comparison of secreted peptides and proteins.

Embodiment 69: A method for studying the effects of mass-tag labeled cellular secretome components on recipient cells' phenotype and function, comprising the internalization of mass-tag labeled components, including a protein from an extracellular vesicle or a virus or a soluble protein, by recipient cells and subsequent analysis using mass cytometry, imaging mass cytometry, electron microscopy, or other mass spectrometry-based single-cell and imaging techniques.

Embodiment 70: The method of embodiment 69, wherein the internalization of mass-tag labeled components of the cellular secretome by recipient cells is not hindered by the integration of the mass tag, and the protein's binding and phenotype remain unaltered.

Embodiment 71: A computer-readable medium containing a non-transitory program for analyzing mass-tag labeled cellular secretome data generated by mass cytometry, imaging mass cytometry, electron microscopy, or other mass spectrometry-based single-cell and imaging techniques, the program comprising instructions for processing, analyzing, and visualizing mass-tag labeled cellular secretome components and their interactions with recipient cells.

Embodiment 72: A method of purifying mass-tag labeled extracellular vesicles, soluble proteins, and viruses from the cellular secretome, comprising a combination of density gradient centrifugation, size-exclusion chromatography, or affinity purification techniques to isolate mass-tag labeled components with minimal contamination of other cellular components.

Embodiment 73: The method of embodiment 72, wherein the purification of mass-tag labeled cellular secretome components facilitates the identification and quantification of specific subpopulations of extracellular vesicles, soluble proteins, and viruses, enhancing the understanding of their roles in intercellular communication.

Embodiment 74: A method for multiplexed analysis of mass-tag labeled cellular secretome components from multiple cell types or treatment conditions, allowing simultaneous comparison of different experimental groups to identify changes in the secretome composition.

Embodiment 75: The method of embodiment 74, wherein the multiplexed analysis can be used to identify specific cellular secretome components that are modulated in response to different stimuli or in various disease states.

Embodiment 76: A method for validating the function of mass-tag labeled cellular secretome components, comprising functional assays to assess the impact of these components on recipient cell signaling pathways, gene expression, and overall cellular function.

Embodiment 77: The method of embodiment 76, wherein the functional validation assays can be used to determine the biological relevance of mass-tag labeled cellular secretome components in the context of intercellular communication and disease pathogenesis.

Embodiment 78: A method for assessing the impact of pharmacological agents on the composition and function of the mass-tag labeled cellular secretome, facilitating drug target identification and the evaluation of drug efficacy and safety.

Embodiment 79: A method for identifying novel mass-tag labeled cellular secretome components with potential therapeutic applications, utilizing high-throughput screening approaches to test the effects of these components on various disease models.

Embodiment 80: The mass-tag labeled cellular secretome of embodiment 61, for use in the development of diagnostic tools to monitor disease progression or treatment response by measuring changes in the composition and function of the cellular secretome.

Embodiment 81: A method for generating mass-tag labeled cellular secretome libraries for the purpose of high-throughput screening, enabling the identification of novel secretome components with potential therapeutic or diagnostic applications.

Embodiment 82: A method for optimizing the mass-tag labeling process, comprising the systematic evaluation of labeling efficiency, signal-to-noise ratio, and impact on cellular secretome component function, to ensure the reliability and reproducibility of the labeling method.

Embodiment 83: A method for integrating the mass-tag labeled cellular secretome data with other omics data, such as transcriptomics, genomics, and metabolomics, to provide a comprehensive understanding of the molecular mechanisms underlying intercellular communication and disease pathogenesis.

Embodiment 84: The method of embodiment 56, wherein the mass-tag labeling is adaptable for use with various cell types and organisms, facilitating the study of the cellular secretome in diverse biological systems.

Embodiment 85: A method for tracking the biodistribution and localization of mass-tag labeled cellular secretome components in vivo, employing imaging techniques such as magnetic resonance imaging (MRI), positron emission tomography (PET), or single-photon emission computed tomography (SPECT) to monitor the mass-tag labeled secretome components in living organisms, enabling the assessment of their biological roles and potential therapeutic applications.

### BRIEF DESCRIPTION OF THE DRAWINGS

FIG. 1 shows a schematic representation of an illustrative embodiment of an EV mass-tagging procedure and application examples of the mass-tag labeling of extracellular vesicles and other protein-containing components of the secretome of cells cultured in the presence of TePhe. TePhe can be applied as a media supplement before collecting cell- or cell line-conditioned (CCM) medium. The harvested CCM can be subjected to separation and concentration methods (filtration, size-exclusion chromatography, density gradient centrifugation, tangential flow filtration, ultrafiltration, e.g.), yielding purified and mass-tagged components of the cellular secretome (EVs, virus, secreted proteins, intracellular pathogens) used for tracking by mass cytometry and mass cytometry imaging [11].

FIG. 2 shows the results of transmission electron microscopy analysis with uranyl acetate (UA) contrasting of mass-tagged EVs and untagged controls. No apparent changes in size, morphology, or quantity were observed between mass-tagged and untagged EVs.

FIG. 3 shows the result of transmission electron microscopy analysis without uranyl acetate contrasting of mass-tagged HEK293T cell line-derived EVs. Mass-tagged EVs were contrasted by the proteome integration of TePhe. The arrows indicate the lipid bilayer membrane of EVs that is not visible by transmission electron microscopy with uranyl acetate contrasting.

FIG. 4 shows the results of transmission electron microscopy analysis with and without uranyl acetate of mass-tagged and untagged EVs derived from primary CLL cells and the cell lines HEK293T and OSU-CLL. In the absence of UA, only mass-tagged EVs were contrasted and detectable. Compared to UA contrasted EVs, mass-tag contrasted EVs exhibited subvesicular structures of higher resolution and a distinguishable lipid bilayer membrane.

FIG. 5 shows 130Te histograms derived from the mass cytometric analysis of peripheral blood mononuclear cells (PBMCs) incubated in the presence of mass-tagged EVs of four cell lines compared to the same PBMCs incubated without EVs. 1e6 PBMCs were incubated with mass-tagged EVs (cell-equivalents of 2e6 EV-secreting cells conditioning medium for 48 hours). After 16 hours, PBMCs were washed, barcoded, stained, and analyzed by a Helios mass cytometer. According to the 130Te signal (dual counts), TE+EV recipient cells were gated.

FIG. 6 shows contour plots (89Y-CD45 vs. 130Te) derived from the mass cytometric analysis described in FIG. 5. Median dual counts are depicted for PBMCs incubated in the presence of mass-tagged EVs derived from different cell lines. The percentage of EV recipient cells did not correlate with the median 130Te dual counts between various EV sources. Only CD45+ events after the data clean-up were analyzed.

FIG. 7 shows density tSNE plots (tSNE X vs. tSNE Y) derived from the mass cytometric analysis described in FIG. 5. All PBMC samples were merged and clustered according to the expression of 30 MDIPA markers by the optimized tSNE algorithm (Opt-SNE). Upon EV uptake, the CD14+ monocyte cluster (heterogeneous bottom island) displayed phenotypic changes pronounced in PBMCs incubated with mass-tagged OSU-CLL and HeLa EVs.

FIG. 8 shows density tSNE plots from FIG. 7 with a 130Te signal heatmap overlay. The phenotypic changes in the monocytic cell cluster correspond to the EV uptake depicted by the 130Te signal.

FIG. 9 shows a FlowSOM cell cluster heatmap derived from the mass cytometric analysis of PBMCs incubated in the presence of mass-tagged primary CLL EVs. 1e6 PBMCs were incubated with mass-tagged EVs (cell-equivalents of 2e6 EV-secreting cells conditioning medium for 48 hours). After 16 hours, PBMCs were washed, barcoded, stained, and analyzed by a Helios mass cytometer. Cells were clustered according to the expression of 30 MDIPA markers by the FlowSOM algorithm. All lineage markers are depicted as normalized arithmetic means in the heatmap to identify each cell cluster. The first column displays the 130Te signal linking EV uptake to each cell cluster. Cluster 5 exhibited CD14 and CD11c expression and was consequently identified as the monocyte cluster, demonstrating the highest EV uptake.

FIG. 10 shows a density tSNE plot from the experiment with mass-tagged primary CLL EVs according to FIGS. 7 and 9. The FlowSOM algorithm-derived cluster 5 is color-overlayed in black and marks the CD11c+ and CD14+ monocyte island with the highest EV uptake.

FIG. 11 shows a novel mass cytometric panel for PBMC analysis which is useful for simultaneously identifying EV uptake in more than 30 cell populations and analyzing effects on key signaling pathways. More specifically, FIG. 11 shows the human mass cytometry panel employed for the identification of cellular secretome components, functional cellular markers, and cell lineage markers in peripheral blood mononuclear cell (PBMC) recipient cells.

FIG. 12 illustrates an example of mass cytometric gating of PBMC subpopulations and live-cell barcoding and was partially created with Biorender.com. More specifically, FIG. 12 shows the manual gating strategy for PBMC recipient cells, highlighting the benefits of utilizing a combined CD45-Cd live cell barcoding approach.

FIG. 13 demonstrates that tellurium-based labeling of the EV proteome (TeLEV) did not change the particle or protein concentration, the size, or the morphology of EVs. More specifically, FIG. 13 shows the characterization of cellular secretome components in compliance with MISEV2018 guidelines through micro bicinchoninic acid assay (micro BCA), nanoparticle tracking analysis (NTA), and transmission electron microscopy (TEM). Mass-tag labeled extracellular vesicles (EVs) and soluble proteins exhibit no differences in protein or particle concentration as measured by microBCA or NTA, respectively. The cup-shaped morphology of mass-tag labeled EVs in TEM is consistent with control EVs.

FIG. 14 demonstrates that TeLEV did not change the expression of EV markers and did not alter the MISEV2018 characteristics of the EV samples. More specifically, FIG. 14 shows the characterization of cellular secretome components via immunoblotting of size-exclusion chromatography (SEC) fractions and immunogold transmission electron microscopy of EVs. Mass-tag labeled components of the cellular secretome (SEC fractions) exhibit no differences in the presence of positive EV markers (CD81, Flotillin-1, Syntenin-1) or the absence of negative markers (Calnexin). Mass-tag labeled and control EVs both demonstrate tetraspanin expression of CD63 and CD81 and a cup-shaped morphology in immunogold transmission electron microscopy.

FIG. 15 shows the results of a study in which healthy donor PBMCs were incubated with primary CLL TeLEV SEC fractions for 16 hours. TePhe labeled all protein-containing components of the cellular secretome, enabling a direct comparison between EV and soluble protein-mediated effects at a single-cell level. More specifically, FIG. 15 shows a t-distributed Stochastic Neighbor Embedding (tSNE) representation of PBMC recipient cells of mass-tag labeled components of the cellular secretome of primary chronic lymphocytic leukemia (CLL) cells with a heatmap overlay of the 130Te signal. Arrows indicate differential Te signal patterns between EV fractions (Fraction 1-3) and soluble protein fractions (Fraction 8-10).

FIG. 16 show the results of a study in which healthy donor PBMCs were incubated with different sources of TeLEVS for 16 hours. More specifically, FIG. 16 shows cell analyses of recipient cells of mass-tag labeled components of the cellular secretome (extracellular vesicles) from six different production cell lines and from primary CLL cells. Regardless of the production cell, EV uptake is highest in myeloid dendritic cells and monocyte subsets. The recipient cell pattern signatures of 130Te differ between the respective cell types, indicating EV uptake specificity.

FIG. 17 shows CD9-immunogold-labeled EVs in transmission electron microscopy. EVs are contrasted only by their mass-tag label and the EV membrane (black arrows), which can be differentiated into three parts due to the tellurium-based contrast (dark-light-dark).

FIG. 18 shows recipient cell analysis by transmission electron microscopy. Recipient cells of the components of the cellular secretome exhibit aggregates of mass-tag labeled EVs and soluble proteins (black arrows) that are contrasted by mass-tag integration.

FIG. 19 shows recipient cell analysis exposed to all SEC fractions of the cellular secretome derived from genetically modified HEK293T cells. The 130Te signal between PBMC cell types is specific for different secretome components.

FIG. 20 shows extended FlowSOM PBMC sub-clustering analyses based on FIG. 19. FlowSOM meta clusters from the left panel are depicted in the right panel for their 130Te signal. Each row represents recipient cells of distinct SEC fractions of the cellular secretome.

FIG. 21 shows the extended tSNE-based density analysis of FIG. 19. The density map in the right panel depicts phenotypical changes of the PBMC recipient cells upon uptake of distinct SEC secretome fractions.

FIG. 22 shows imaging mass cytometric analyses of recipient cells of EV and soluble protein components of the cellular secretome from primary CLL cells, OSU-CLL cell line, and HEK293T cell line.

FIG. 23 shows the deep immunophenotyping of PBMC recipient cells based on the data presented in FIG. 22.

FIG. 24 shows the tellurium-based biodistribution of components of the cellular secretome in PBMC subpopulations as derived from FIGS. 22-23.

FIG. 25 shows a tSNE representation of PBMC recipient cells based on the data presented in FIGS. 22-24.

FIG. 26 shows ex vivo biodistribution analyses of mass-tag labeled components of the cellular secretome in murine C57BL/6J wild-type recipient splenocytes, along with the simultaneous analysis of CD62L and CD8a expression.

FIG. 27 shows in vivo biodistribution analyses of mass-tag labeled components of the cellular secretome one hour after tail vein injection into mice.

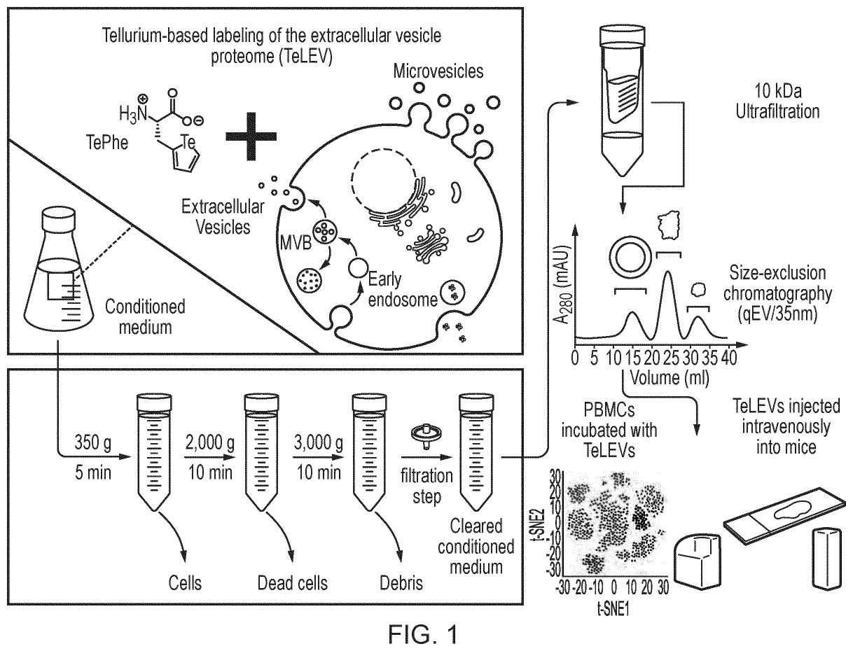

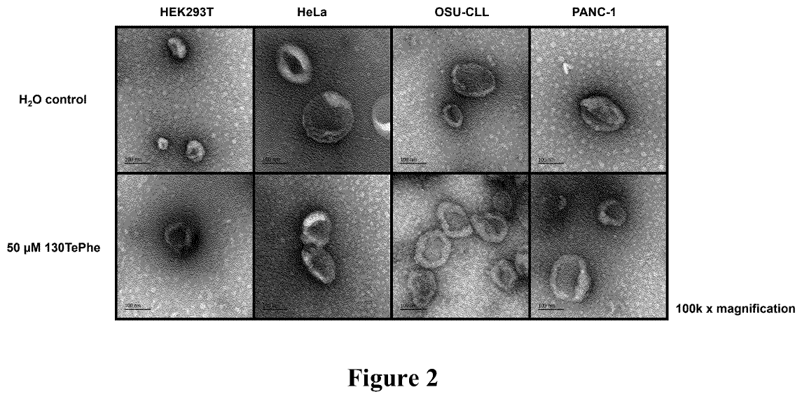

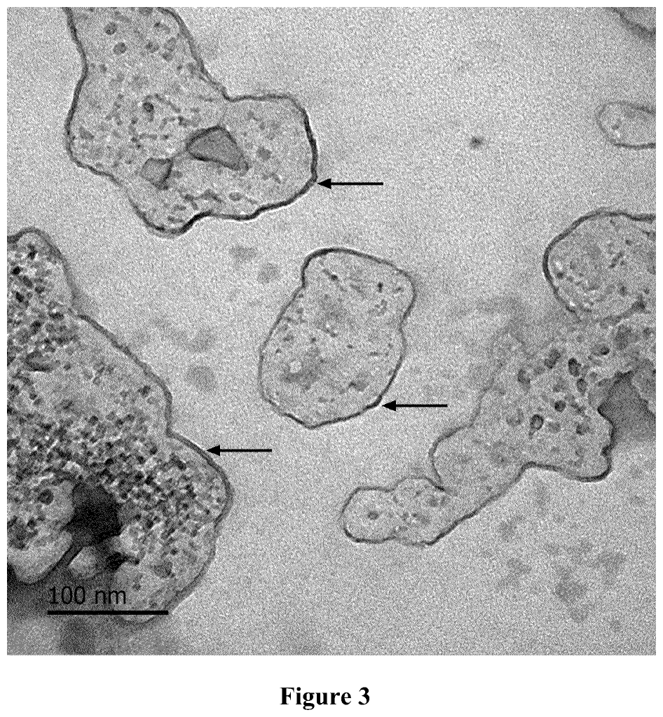

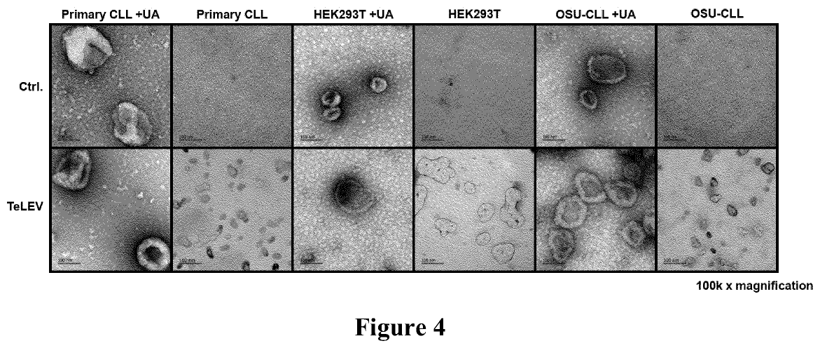

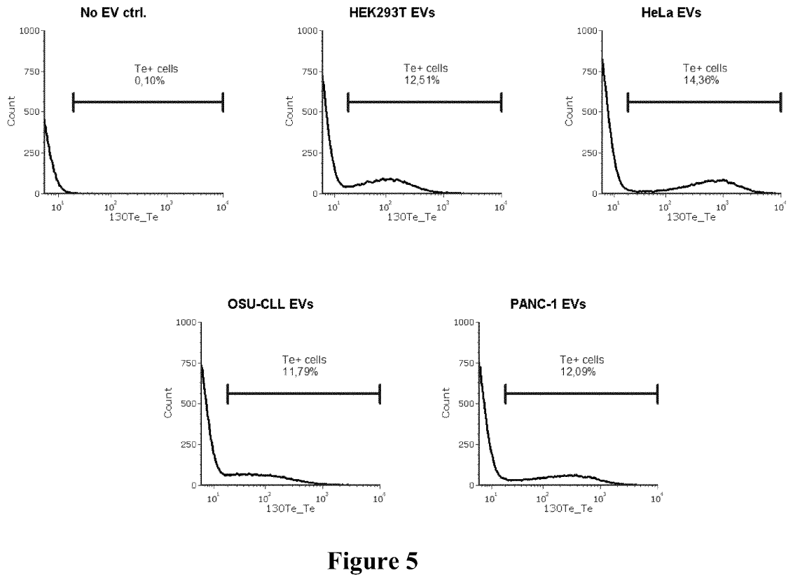

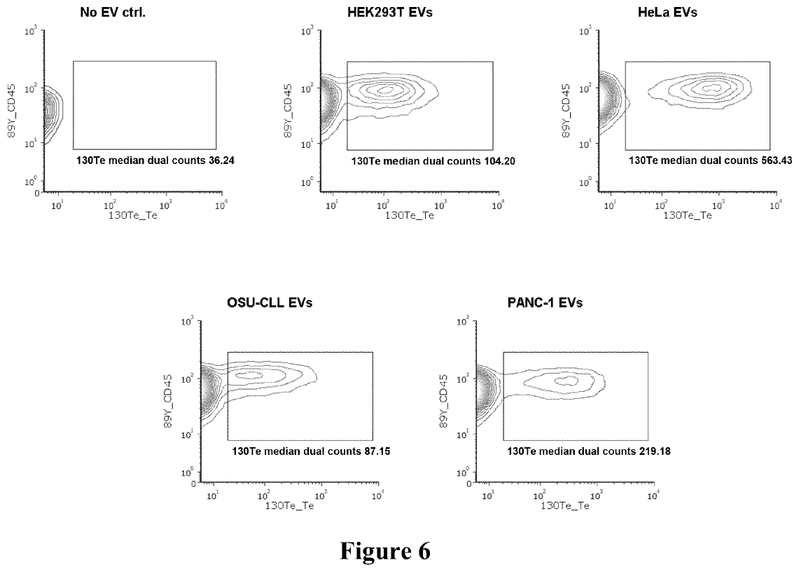

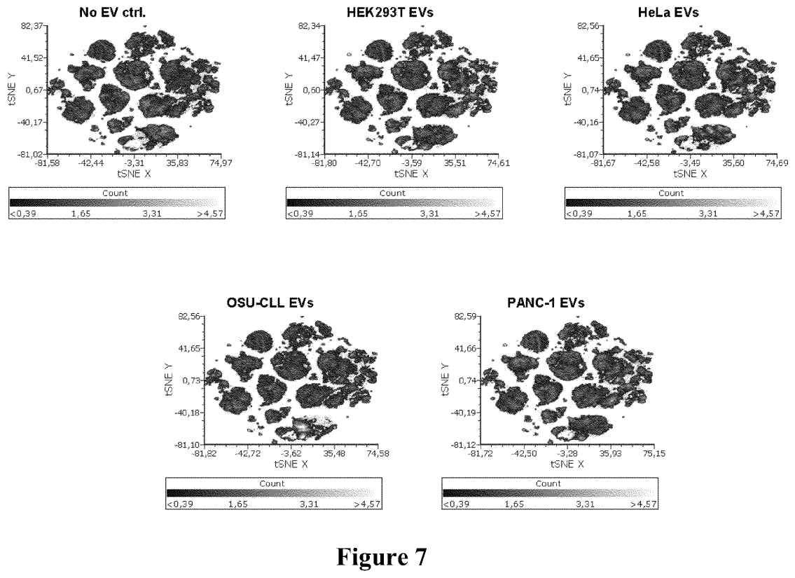

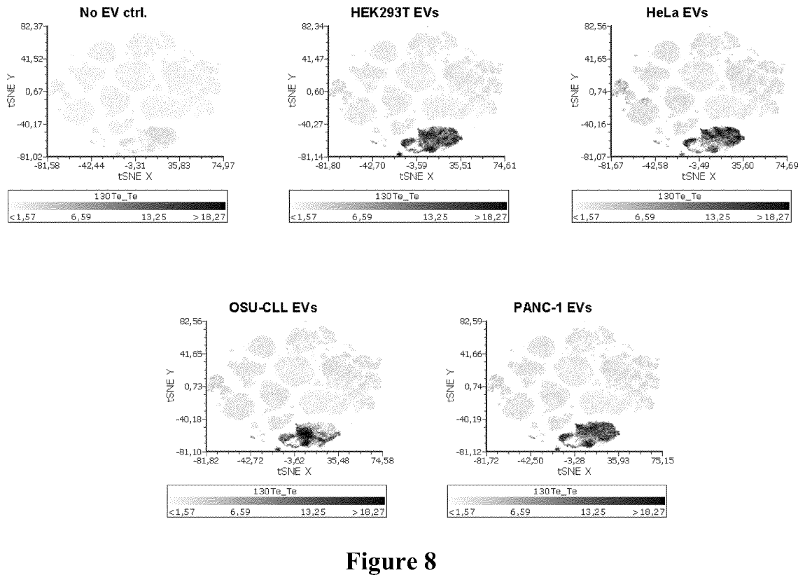

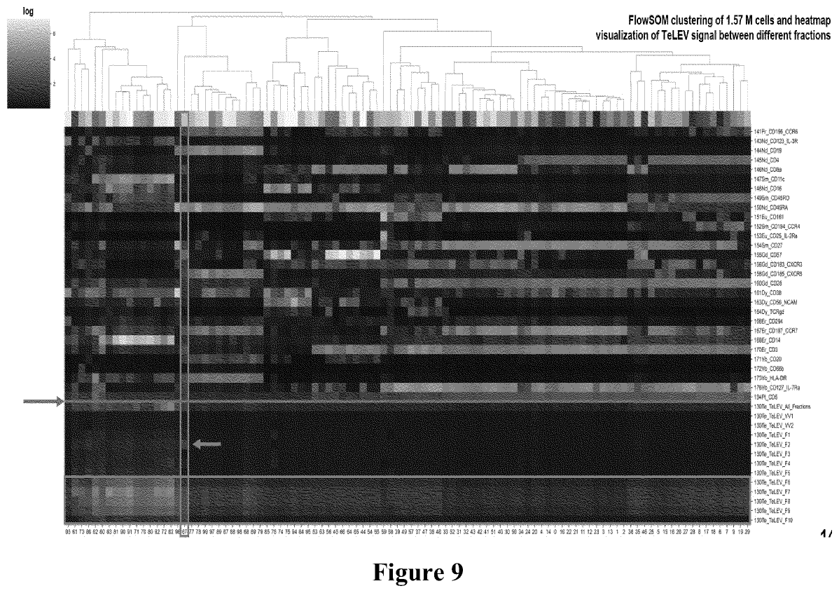

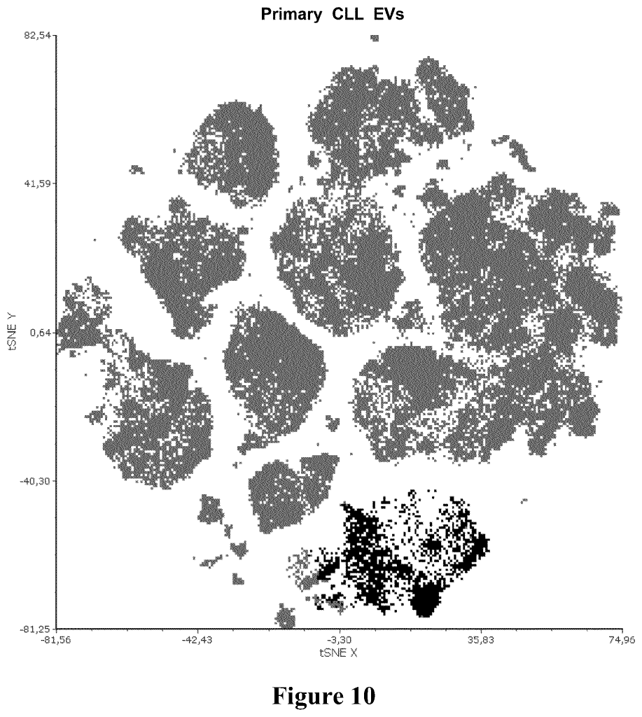

### DETAILED DESCRIPTION

### Definitions

Terms used in the claims and specification are defined as set forth below unless otherwise specified.

The term “soluble component” is used herein to refer to any component that is released from, or secreted by, a cell into the extracellular space.

The term “extracellular vesicle” (“EV”) is used herein to refer to membrane vesicles released or shed by cells into the extracellular environment. Examples include, but are not limited to, microvesicles, exosomes, ectosomes, argosomes, and apoptic bodies. They can be classified into different subtypes based on their biogenesis, size, and composition. EVs contain a variety of biological molecules, including proteins, lipids, and nucleic acids, which can be transferred between cells to mediate intercellular communication, regulate immune responses, and contribute to disease progression. Due to their involvement in various pathological processes, EVs are being explored as potential diagnostic biomarkers and therapeutic targets.

A used herein, the term “mass tag” refers to a molecule that includes at least one specific elemental isotopic composition that serves to distinguish a molecule to which the tag is attached, or optionally the tag itself, from other molecules including a different elemental isotopic composition using a mass spectral analysis. In some embodiments, the mass tag comprises at least one elemental isotope and a supporting structure for the at least one elemental isotope.

The term “proteome” is used herein to refer to the entire complement of proteins expressed in an organism, tissue, cell, or sub-cellular component (such as, e.g., an EV). The make-up of the proteome can vary, depending on specific time or conditions. It is thus a dynamic entity, as protein expression levels and modifications can change in response to environmental factors, developmental stages, and disease states. Proteomics, the large-scale study of proteomes, aims to identify, quantify, and characterize proteins to better understand their functions, interactions, and roles in cellular processes and disease mechanisms. Components of the cellular proteome comprise the cellular secretome and all intracellular proteins that are not secreted (intracellular proteome).

As used herein, the term “secretome” is used herein to refer to all factors secreted into the extracellular space by a cell or sub-cellular component there of (such as, e.g., an EV). These factors include proteins, and the term “cellular secretome” is often used in the art to describe the collection of proteins that are secreted by a cell into its surrounding extracellular environment. These proteins can include growth factors, cytokines, hormones, other signaling molecules, and the extracellular vesicle proteins (extracellular vesicle proteome). The secretome plays a critical role in cell-to-cell communication, the regulation of cellular processes, and the maintenance of tissue homeostasis.

“Secreted soluble proteins” are a subset of proteins within the secretome that are released into the extracellular environment in a soluble form. They can diffuse freely through the extracellular space and interact with other cells, receptors, or molecules to exert their functions. Examples of secreted soluble proteins include cytokines, chemokines, growth factors, antibodies and hormones, which play vital roles in regulating immune responses, cell growth and differentiation, and tissue repair and remodeling.

As used herein, the term “purify” refers to separating a desired component from at least one other component with which it found. The term encompasses all degrees of purification, including purification to a level wherein the desired component is present in a purified composition at levels of 50, 55, 60, 65, 70, 75, 80, 85, 90, 95, 96, 97, 98, 99, or 100 percent.

The term “production cell” is used herein to refer to a cell that produces a secretome or a component thereof, including EVs, viruses and soluble proteins and peptides.

The term “recipient cell” is used herein to refer to a cell that takes up one or more components of a secretome, such as an EV, virus, and/or a soluble protein and/or peptide.

The term “detectable label” or “label,” as used herein, refers to any atom or molecule that can be used to provide a detectable and/or quantifiable signal. In particular, the label can be attached, directly or indirectly, to a nucleic acid or protein. Suitable labels that include, but are not limited to, radioisotopes, fluorophores, chromophores, mass labels, electron dense particles, magnetic particles, spin labels, molecules that emit chemiluminescence, electrochemically active molecules, enzymes, cofactors, and enzyme substrates.

As used herein, the term “barcoding” is used to refer to labeling an entity (e.g., a cell) with a detectable label or a combination of detectable labels that encode information. For example, a barcode can identify, e.g., the source or type of a biological entity, such as a cell. Barcodes can be used, e.g., to distinguish different samples, different cells, different treatments, different time points, etc.

As used with reference to an analysis, the term “multiplex” refers to the situation in which multiple, separate analyses are conducted together in one “pool,” that is subjected to a single set of conditions. For example, multiplex analysis enables a single study to be performed with multiple samples, each of which is distinguishable from every other sample. This ability to distinguish between samples enables the results of the study to be deconvoluted such that particular results can be assigned to particular samples. Multiplex analysis offers the advantage over single-plex analysis in that artifactual differences in results arising from conducting single-plex in parallel (e.g., due to unavoidable differences in samples or conditions in separate single-plex analyses) can be eliminated.

The term “antibody” as used herein, is intended to include monoclonal antibodies, polyclonal antibodies, and chimeric antibodies and binding fragments thereof, as well as antibodies of atypical structure from species like camelids and shark (e.g., nanobodies). The antibody can be from recombinant sources and/or produced in transgenic animals. Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be synthesized by recombinant techniques. Antibody fragment, as used herein refers to a fragment that binds a target antigen.

The term “amino acid residue” as used herein refers to natural, synthetic, or modified amino acids. Various amino acid analogues include, but are not limited to 2-aminoadipic acid, 3-aminoadipic acid, beta-alanine (beta-aminopropionic acid), 2-aminobutyric acid, 4-aminobutyric acid, piperidinic acid, 6-aminocaproic acid, 2-aminoheptanoic acid, 2-aminoisobutyric acid, 3-aminoisobutyric acid, 2-aminopimelic acid, 2,4 diaminobutyric acid, desmosine, 2,2′-diaminopimelic acid, 2,3-diaminopropionic acid, N-ethylglycine, n-ethylasparagine, hydroxylysine, allo-hydroxylysine, 3-hydroxyproline, 4-hydroxyproline, isodesmosine, allo-isoleucine, n-methylglycine, sarcosine, n-methylisoleucine, 6-n-methyllysine, n-methylvaline, norvaline, norleucine, ornithine, L-2-amino-3-guanidinopropionic acid (AGP), L-α,γ-diaminobutyric acid (DAB), L-α,β-diaminoproprionic acid (DAP), L-α-t-butylglycine and the like. These modified amino acid residues are illustrative and not intended to be limiting.

The term “tellurophene” as used herein refers to a compound of the formula:

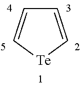

wherein the numbers are used in the naming of various substituents on the tellurophene ring.

The term “organotellurophene” refers to a tellurophene substituted with at least one carbon-containing group.

As used herein, “organotellurophene tag” refers, in some embodiments, to any tellurophene-containing compound comprising a tellurophene moiety and a linker that is for example compact and can be conjugated to biosensor, a polymeric backbone, a biologically active material, and/or another molecule, such as an amino acid, and includes for example an organotellurophene compound described herein and/or described in PCT Pub. No. WO 2016/0206046, which is hereby incorporated by reference for this description. For example, the organotellurophene tag can comprise a tellurophene moiety and a chemical bond or linker conjugating that moiety to another entity, either directly or indirectly. In some embodiments, an entity may be tagged with an organotellurophene tag by substituting the tellurophene group for another moiety in the entity, as shown below, where a phenyl group is substituted with an organotellurophene analog. For example, organotellurophene-substituted phenylalanine can be synthesized as L-enantiomer (>95%) in four steps from N-Boc-L-propargylglycine: adding to (Bromoethynyl)triisopropylsilane resulting in intermediates (S)-2-((tert-butoxycarbonyl)amino)-7-(triisopropylsilyl)hepta-4,6-diynoic acid and (S)-2-((tert-butoxycarbonyl)amino)-3-(tellurophen-2-yl)propanoic acid and finally converting to TePhe (L-2-tellurienylalanine). (See Bassan, J., et al. (2019) Proc. Natl. Acad. Sci. USA 116(17):8155-8160, which is incorporated by reference herein for its description of the synthesis and use of an organotellurophene-substituted phenylalanine; and Vurgun, N., and Nitz, M. (2019) Chem. Europe 21(8): 1136-1139, which is incorporated by reference herein for its description of the synthesis and use of an organotellurophene-substituted phenylalanine.) Organotellurophene tags can include distinct tellurium isotopes for labeling different entities, which enables distinct detection of each, e.g., in multiplex analyses.

L-2-tellurienylalanine, also referred to herein as TePhe, is an organotellurophene substituted phenylalanine, which has the chemical structure shown in Formula 1:

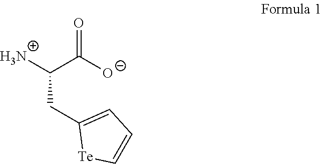

N-(2-(2,5-dioxo-2,5-dihydro-1H-pyrrol-1-yl)ethyl)-3-(tellurophen-2-yl)propenamide, also referred to herein as TeMal, is an thiol-reactive maleimide functionalized tellurophene, which has the chemical structure shown in Formula 2:

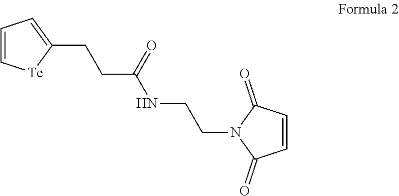

The term “distinct tellurium isotope,” as used herein, refers to Te atoms in a compound having one or more atoms of a single tellurium isotope. For example, a series of mass tagged entities can be employed in an assay each having a different distinct tellurium isotope, such that each compound comprising a distinct tellurium isotope is distinguishable from other compounds.

As used herein, an “isotopologue” is a chemical that differs from its parent chemical in that at least one atom has a different number of neutrons.

The term “distinct mass,” as used herein, indicates that the compound has one or more atoms of a single tellurium isotope or a unique combination of tellurium isotopes alone (e.g. distinct tellurium mass) or in combination with other mass tags. An example includes a series of compounds, optionally polymers, each with different levels of different tellurium isotopes alone or combined with other mess tags, optionally for use in barcoding embodiments.

The term “metabolic labeling,” as used herein, refers to incorporation of a labeled molecule into another molecule, for example, a macromolecule, as in e.g., the incorporation of an amino acid into a protein or a monosaccharide into a polysaccharide or glycoprotein. Metabolic labeling is typically carried out by a cell, or metabolically active component thereof, that is exposed to the labeled molecule. Metabolic labeling is a technique that can be used to study cellular turnover and trafficking within cells. In some embodiments, it involves the incorporation of labeled amino acids (e.g., with isotopes, biotin, or fluorescent tags) into newly synthesized proteins during a defined time period. By tracking the labeled amino acids, researchers can monitor the synthesis, degradation, localization, and interaction of proteins in living cells or organisms. Metabolic labeling has been widely used in proteomics research to quantify protein abundance, assess protein-protein interactions, and investigate post-translational modifications.

The term “functional group,” as used herein, refers to a group of atoms or a single atom that will react with another group of atoms or a single atom (a so-called “complementary functional group”) to form a chemical interaction, typically a chemical bond, between the two groups or atoms. A moiety such as a mass tag or other label can be functionalized for conjugation or binding with a molecule such, e.g., as a component that can be taken up by a cell (e.g., for metabolic labeling) or an antibody that binds to a cell-surface antigen to label the surface of a cell.

As used herein the phrase “effect(s) on a recipient cell” of an EV or other soluble component of the cellular secretome refers to any effect on any function of a cell observed after EV or soluble component contact with, and/or uptake by, the cell, including, but not limited to, any change is cellular function (e.g., signaling) that can be measured using mass cytometry or mass cytometry imaging or to one or more or all MISEV2018 characteristics.

As used herein, the words “comprising” (and any form of comprising, such as “comprise” and “comprises”), “having” (and any form of having, such as “have” and “has”), “including” (and any form of including, such as “include” and “includes”) or “containing” ” (and any form of containing, such as “contain” and “contains”), are inclusive or open-ended and do not exclude additional, unrecited elements or process steps.

As used herein, the term “consisting” and its derivatives, are intended to be closed-ended terms that specify the presence of stated features, elements, components, groups, integers, and/or steps, and also exclude the presence of other unstated features, elements, components, groups, integers and/or steps.

The term “consisting essentially of,” as used herein, is intended to specify the presence of the stated features, elements, components, groups, integers, and/or steps as well as those that do not materially affect the basic and novel characteristic(s) of these features, elements, components, groups, integers, and/or steps. The basic and novel characteristic(s) of mass-tagged EVs are the presence of a mass tag in combination with 1, 2, 3, 4, 5, 6, 7, 8, 9, or 10 or more characteristics that do not differ substantially from the characteristic(s) of non-mass-tagged EVs of the same type.

The terms “about,” “substantially,” and “approximately,” as used herein mean a reasonable amount of deviation of the modified term such that the end result is not significantly changed. These terms of degree should be construed as including a deviation of at least ±20% of the modified term if this deviation would not negate the meaning of the word it modifies. Thus, for example, “substantially similar effect(s) or “substantially the same effects” on a recipient cell refers to one or more effects on a recipient cell, typically elicited by EVs or other components of the cellular secretome that differ by no more than ±20%. In some embodiments, higher degrees of similarity are observed, such as, e.g., at least ±15, ±14, ±13±12, ±11, ±10, ±9, ±8, ±7, ±6, ±5, ±4, ±3, ±2, ±1, ±0.5% percent or a degree of similarity falling within any range bounded by any of these values, for example ±5 to ±0.5 percent. As used with reference to mass tagged EVs, as compared to unlabeled EVs, more generally, “substantially similar” and “substantially the same” refer to the similarity of EVs as mass-tagged compared to unlabeled as determined by accepted methodology in the field, for example as set out in MISEV2018 Guidelines, including: morphology, physical and biochemical characteristics, protein concentration, EV concentration, cell type specificity of uptake, size distribution, purification behavior, distributions of EV components (including protein, RNA, lipids, polysaccharides and glycans) and other general properties/behaviors set out in MISEV2018 Guidelines. The degrees of similarity discussed above with respect to “effect(s)s” apply equally to the more general aspects of EVs, including those set forth in the MISEV2018 Guidelines.

### Mass-Tagging of the Cellular Secretome

The present disclosure provides methods of mass-tagging one or more soluble components of the cellular secretome, which can include, e.g., an extracellular vesicle (EV), a virus particle, a cellular secretome, an EV proteome or secretome, or a component of any of these. In certain embodiments, this method entails exposing a cell termed “a production cell” to a mass-tag-labeled component. In some embodiments, the component is one that can be taken up by the production cell and incorporated by the cell into a molecule produced by the cell, a method termed “metabolic labeling.” In some embodiments, the molecule produced by the cell is secreted or otherwise released, either by itself or as part of a more complex structure, such as an EV; in either case, the production cell produces a mass-tagged soluble component. In some embodiments, the method entails purifying the mass-tagged soluble component. In other embodiments, a mass-tagged soluble component is produced by directly tagging the soluble component. However, it is produce, the mass-tagged soluble component can then be used to study its binding, uptake, and or biological effects in a biological system of interest, which can include a tissue, and organ, or cells in vitro or in vivo. Mass-tagged soluble components can be used in conjunction with other labeling strategies (including distinct mass tags) to characterize, e.g., the biological effects of the soluble component, as well as the tissues, organs, or cells affected. These studies can be carried out at the single-cell level.

In order to effectively compare various components of the cellular secretome, it is essential, in some embodiments, to employ an intrinsically normalized labeling technique. The mass-tag labeling method for the cellular secretome achieves this by randomly substituting Phe with TePhe (metabolic labeling of the cellular secretome) in all proteins and peptides within the secretome. This process follows a Gaussian distribution, ensuring consistent and uniform labeling across the entire cellular secretome. Contrarily, prior labeling methods lack these characteristics, rendering them unsuitable for facilitating direct comparisons between different secretome components.

The novel mass-tag labeling approach for the cellular secretome has broad implications for various areas of biotechnology and biomedical research, including but not limited to:

Biomarker discovery: The method can be used to identify novel biomarkers for disease diagnosis, prognosis, and treatment response monitoring.

Drug target identification and biodistribution: By enabling a more comprehensive understanding of the cellular secretome, our method can facilitate the discovery of new cellular drug targets and provide insights into the biodistribution of drugs for a wide range of diseases.

Basic research: The proposed mass-tag labeling technique can provide valuable insights into the molecular mechanisms of intercellular communication and function, advancing our understanding of fundamental biological communication processes.

### Mass Tags

Mass cytometry reagents are tagged with metal isotopes of defined mass that act as labels. Metals can be detected using inductively coupled plasma time-of-flight mass spectrometry (ICP-TOF-MS). Many different types of mass-tag reagents have been developed. These reagents include polymer-based mass-tag reagents, nonpolymer-based mass-tag reagents, and inorganic nanoparticles. Metal-chelating polymers (MCPs) are widely used to profile and quantify cellular biomarkers. Biocompatible materials such as polystyrene and inorganic nanoparticles are also used in mass cytometry. Polystyrene allows the inclusion of a wide variety of metals, whereas the high metal content of inorganic nanoparticles offers an excellent opportunity to increase the signal from the metals to detect low-abundance biomarkers. Nonpolymer-based mass-tag reagents can be used in multiple applications: cell detection, cell cycle property determination, biomarker detection, and mass-tag cellular barcoding (MCB). Recent developments have been achieved in live cell barcoding by targeting proteins (CD45, b2m, and CD298), by using small and nonpolar probes or by ratiometric barcoding. Mass cytometry reagents are reviewed in Delgado-Gonzalez and Sanchez-Martin (2021) Anal. Chem. 93(2):657-664, which is incorporated by reference herein for this description.

Mass tags useful in the methods and compositions described herein have, in some embodiments, one or more, or preferably all, of the following characteristics: accessible in a high-yielding synthesis amenable to isotope incorporation, stable under biologically-relevant conditionsm and low toxicity. A mass tagged probe (Telox) for measuring cellular hypoxia has constructed (described in U.S. Application No. 62/039,762). This probe used a 2-nitroimidazole as the activity group for hypoxic-specific labeling and a methyl telluroether functionality as the tag unit for MC detection. Tellurium was chosen to be the element for detection as it is known to form stable bonds with carbon and it has 8 naturally occurring isotopes that can be accessed to generate a series of uniquely identifiable, biologically indistinguishable mass cytometry (MC) probes using the same chemistry. In Telox, the telluroether functionality had moderate stability and a metabolic LD50 value close to the required assay concentration. The synthesis, aqueous/aerobic stability, and in vitro toxicity of a series of alkyl telluroethers and tellurophene functional groups, as well as their use, is described in detail in PCT Publication No. WO2016026046, which is incorporated by reference herein for this description. Based on the guidance therein, one of skill in the art can label a component with an organotellurophene tag.

Mass tags may be incorporated directly into biological components of interest, e.g., by substituting the tellurophene for a phenyl moiety in the component and/or added to the component via a linker. Biological components may also be labeled directly or indirectly using mass tag-functionalized binding moieties, such as antibodies, streptavidin-biotin, etc. A wide variety of labeling strategies are available and can readily be adapted to the use of mass tags as the detectable label.

### Mass-Tagging Methods

### Tagging Via Metabolic Labeling

Those of skill in the art of cell biology appreciate that a wide variety of components can be taken up by cells and incorporated into other molecules during cellular metabolism. Examples include, but are not limited to, amino acids, sugars, salts, lipids, and elements. The choice of a particular component can, in some embodiments, be informed by the aim to mass-tag a particular aspect of the cell. For example, if the desire is to mass-tag the cell's proteome, an amino acid can conveniently be used to introduce a mass tag into the proteins of the cell. Likewise, the choice of particular amino acid can depend on the stucture of the mass tag. Organotellurophene tags can readily be incorporated into amino acids by subsitution for a phenyl moiety. This approach is illustrated in an Example below with the use of the mass-tagged version of phenylalanine L-2-tellurienylalanine (TePhe).

For metabolic tagging, the cell is exposed to the mass-tagged component under conditions that enable the cell to uptake a sufficent amount of the mass-tagged component to facilitate the desired labeling. This can entail reducing any tendency of the mass-tagged component to be degraded or to aggregate or bind to other components in the cell's milieu that could inhibit the mass tagged-component's uptake and/or subjecting the cell to conditions that promote uptake of the mass-tagged component. For a mass-tagged amino acid, in some embodiments, it may be advantageous to expose the cell to the mass-tagged amino acid under serum-free conditions, as illustrated in the Example below. Those of skill in art are aware of, or can determine empirically, suitable conditions for a particular mass-tagged component, cell, and cellular aspect to be mass-tagged.

Any cell that can take up and incorporate a mass-tagged component can be employed in this metabolic labeling approach to mass tagging. In the case of TePhe, for example, the cell preferably has some means of taking up Phe, such as, e.g., a Phe transporter. Suitable cells include those from bacteria, protozoa, fungi, as well higher organisms such as plants or animals, particularly mammals, and more particularly humans. Metabolic labeling with mass tags can be carried out on cell lines (e.g., HEK293T, HeLa, OSU-CLL, and PANC-1), primary cells (e.g., chronic lymphocytic leukemia cells), and in vivo. The only requirements are that the cell be viable and capable of incorporating the mass-tagged component.

### Direct Tagging

In some embodiments, a soluble component of interest, such as, e.g., an extracellular vesicle (EV) or a virus particle can be tagged directly, by contacting the soluble component with a mass tag that is functionalized to bind to a feature of the soluble component. Such binding can be of any type, e.g., covalent or non-covalent, and can be direct or indirect. In some embodiments the soluble component (e.g., EV) is purified from a bodily fluid or tissue of an organism prior to tagging.

For example, mass-tag labeling of EVs derived from solid tissues or biological fluids can be directly performed with TeMal isotopologues after size-exclusion chromatography-based separation and ultrafiltration-based concentration of the EV fractions. See, for example, Willis, L. M., et al. (2018) Tellurium-based mass cytometry barcode for live and fixed cells,” Cytometry, https://doi.org/10.1002/cyto.a.23495 (which is incorporated by reference herein for its description of direct tagging with TeMal). In an illustrative embodiment, TeMal at a total concentration of 6 μM can be added to a concentrate of EV-containing fractions (200 μL) after 10 kDa MWCO ultrafiltration. Next, the concentrate of EVs can be incubated with TeMal for 30 minutes at room temperature within the ultrafiltration unit and afterward washed twice (4° C., 20 min, 3000 RCF) with 14.8 mL and 15.0 mL PBS, respectively. After the last washing step, mass-tagged samples are ready to be used in mass cytometric and imaging mass cytometric assays.

Other labeling strategies that may be useful in conjunction with the methods described herein Qin, W., et al. (2021) “Deciphering molecular interactions by proximity labeling,” Nat. Methods 18:133-143 (which is hereby incorporated by reference herein for this description); and Sufi, J., et al. (2021) “Multiplexed single-cell analysis of organoid signaling networks,” Nat. Protoc. 16(10):4897-4918 (which is hereby incorporated by reference herein for this description).

### Nucleic Acid Tagging

In some embodiments, the cellular aspect tagged are the cell's nucleic acids. Suitable components for mass tagging to label nucleic acids are known to those of skill in the art and include, for example, nucleic acid binders or DNA intercalators, such as cisplatin, iridium, or rhodium. These components can be introduced into a cell or into EVs by standard techniques, such as transfection or electroporation. See, for example, Wang, J., et al. (2020) “Loading of metal isotope-containing intercalators for mass cytometry-based high-throughput quantitation of exosome uptake at the single-cell level,” Biomaterials 255:120152 (which is hereby incorporated by reference herein for this description). One caveat associated with this approach is that the nucleic acid cargo of EVs can vary quite a bit at a single-EV level, and it is not proven that every EV caries nucleic acids. Also, transfection and electroporation can alter membrane structure and thus, for example, EV function. Metabolic labeling avoids both of these issues.

### Purification of Mass-Tagged Secretome Components

Mass-tagged secretome components can generally be purified using essentially the same techniques as used for their non-tagged counterparts, and suitable purification protocols can be devised by those of skill in the art, depending on the nature of the soluble component.

Various purification techniques that may be employed include filtration, membrane crossflow ultrafiltration or microfiltration, tangential flow ultrafiltration or microfiltration or dead-end filtration, centrifugation (e.g., density gradient centrifugation), decantation. Additional steps may include one or more of the following: nanofiltration, concentration, extraction, crystallization, precipitation, adsorption, and chromatography (e.g., size-exclusion or ion-exchange).

In a particular embodiment, illustrated in the Example below, mass-tagged EVs were purified by a combination of filtration, ultrafiltration and size-exclusion chromatography. Specifically, 0.2 μM filtration, followed by 10 kDa ultrafiltration, and then qEV/35 nm chromatography.

### Mass-Tagging of Extracellular Vesicles

Extracellular vesicles (EVs) are membrane-enclosed vesicles released or shed by cells into the extracellular environment. Examples include microvesicles, exosomes, ectosomes, argosomes, and apoptic bodies. Typically, EVs are vesicles of endosomal and/or plasma membrane origin. These EVs can represent, inter alia, a mode of intercellular communication by serving as vehicles for transfer between cells of membrane and cytosolic proteins, lipids, and RNA. The term exosome was initially used for vesicles ranging from 40 to 1,000 nm that are released by a variety of cultured cells (see, e.g., Trams et al. (1981) Biochim. Biophys. Acta. 645: 63-70), but the subcellular origin of these vesicles remained unclear. Later, this nomenclature was adopted for 40-100-nm vesicles released during reticulocyte differentiation as a consequence of multivesicular endosome (MVE) fusion with the plasma membrane (see, e.g., Harding et al. (1984) Eur. J. Cell Biol. 35: 256-263; Pan et al. (1985) J. Cell Biol. 101: 942-948). Later, exosomes were found to be released by B lymphocytes and dendritic cells through a similar route (see, e.g., Zitvogel et al. (1998) Nat. Med. 4: 594-600; Raposo et al. (1996) J. Exp. Med. 183: 1161-1172). Additional cell types of both hematopoietic and nonhematopoietic origin, including, but not limited to cytotoxic T cells, platelets, mast cells, neurons, oligodendrocytes, Schwann cells, and intestinal epithelial cells, have also been shown to release exosomes through MVE fusion with the cell surface (see, e.g., Simons & Raposo (2009) Curr. Opin. Cell Biol. 21: 575-581; Thery E (2009) Nat. Rev. Immunol. 9: 581-593).

EVs are increasingly used as diagnostic tools, and in purified form, they are used therapeutically in a variety of diseases. Clinical trials currently in progress are aimed at testing EV-based therapeutics to treat metastatic pancreatic cancer, colon cancer, and non-small-cell lung cancer. The methods and compositions described herein will facilitate the further development of such applications by providing analytical tools to characterize EVs and their recipient cells. In particular, the method and compositions described herein can help answer questions about where EVs go when they are administered (e.g., organs, spatial tissue distribution, and recipient cell types), as well as the biological effects of therapeutically administered EVs (e.g., proliferation, apoptosis, and intracellular signaling).

Mass-tagged EVs can be produced from any cell that produces a secretome.

In some embodiments, EVs can be tagged with different detectable labels, such as distinct mass tags, e.g., to distinguish one set of EVs from another set. For example, EVs prepared from different production cells and/or from different samples can be mass tagged and then combined into a single pool of EVs, which can then be subjected to further studies, with the advantage that all EVs are exposed to the same study conditions. In certain embodiments, different sets of EVs may be distinguished by different combinations of detectable labels. For example, sets of mass-tagged EVs can have different combinations of mass tags and/or have a common mass tag but one more different detectable labels of a different type (e.g., fluorescent) that allows the unique identification of EVs from a particular set.

These mass tag/labeling strategies discussed with respect to EVs apply equally to any mass-tagged soluble secretome component of interest, including viruses and proteins.

The work described in the Example below demonstrates that mass-tagging of EVs, and in particular of the EV proteome, does not substantially change significant EV characteristics, such as size, morphology, or composition. Thus, surprisingly, mass-tagged EVs do not differ substantially from an unlabeled EV produced from the same cell type under the same conditions as the mass tagged-EVs. In particular, there was no reasonable assurance or expectation, prior to the present work, that mass-tagged EVs would be secreted by production cells, have substantially similar morphology, physical, and biochemical characteristics, be taken up by recipient cells in substantially the same manner as untagged EVs, and have substantially similar effect(s) on one or more cellular functions. Notably, in the work described herein, the mass-tagged EVs and the unlabeled EVs were, for the first time, demonstrated to have substantially the same MISEV2018 characteristics.

Methods of Using Mass-Tagged EVs and/or other Mass-Tagged Components of the Cellular Secretome

Mass-tagged components of the cellular secretome find utility in functional assessments independently of the production cell. For example, antibody-producing hybridoma cells can be contacted with TePhe, as described herein, to puroduce mass-tag labeled monoclonal antibodies, which can be purified and their biodistribution and functional impact on specific cell types analyzed by mass cytometry and imaging mass cytometry. In another example, mass-tagged components of the cellular secretome can be used in combination with magnetic resonance imaging to better elucidate a condition.

Mass-tagged EVs find utility, inter alia, in studies aimed at characterizing EV distribution in a tissue or whole organism, the recipient cells that take up EVs, and EV-mediated changes in cellular behavior. Mass-tagged EVs can simply be contacted with potential recipient cells and, in some embodiments, uptake can be detected by any means of detecting mass tags.

Of particular interest is the study of EV uptake in a heterogenous cell populations. With available means to identify particular cell types, such as labeled antibodies that bind to antigens or combinations of antigens that are characteristic of particular cell types, it is possible to determine which cells in a heterogenous cell population take up particular mass-tagged EVs. If a population of EVs derived from the combination of sets of EVs of different types and bearing different labels (e.g., mass tags) or combinations of labels is contacted with a heterogenous population of cells, it is possible to determine which recipient cell types take up which types of EVs, enabling a multidimensional analysis that was not previously possible with conventional labeling methods.

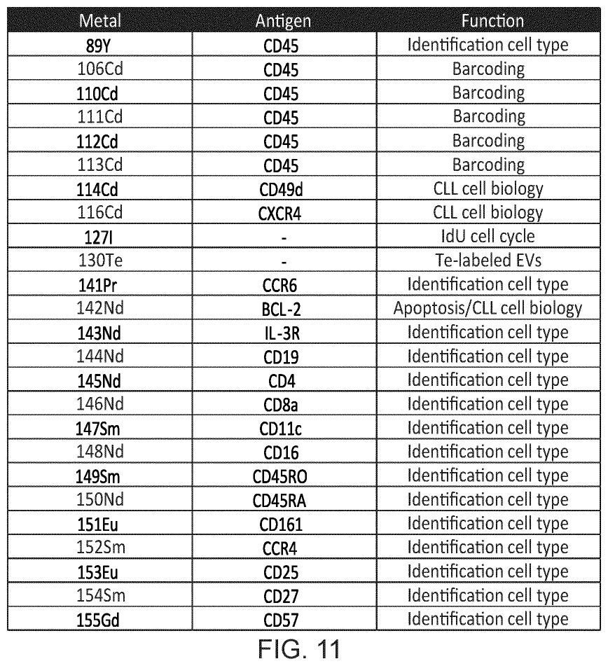

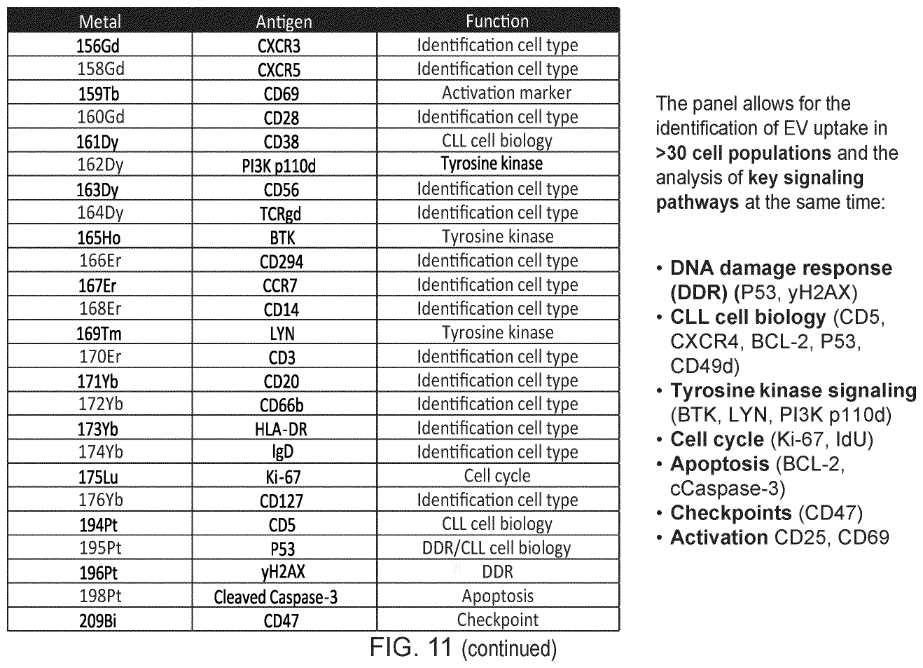

For example, FIG. 11 shows a panel of mass-tagged reagents that allows for the identification of EV uptake in more than 30 different cell types (Standard BioTools' (formerly Fluidigm Corporation) MaxPar® Direct Immune Profiling Assay (MDIPA)). Standard BioTools supports mass cytometry studies with more than 50 metal-tagged reagents, including antibodies, nucleic acid intercalators and analogs, as well as other biochemical ligands. Each reagent is detected and quantified with cytometry by time-of-flight mass spectrometry in Standard BioTools' fully automated CyTOF® system. The high purity and choice of metal isotopes ensure minimal background noise from signal overlap or endogenous cellular components. Standard BioTools' catalog includes with 800 antibodies detecting more than 400 unique human or mouse targets. If a desired antibody is not included, Standard BioTools enables users to make their own using Standard BioTools' metal-labeling kits. Metal-labeled antibodies can also be custom ordered from Standard BioTools.

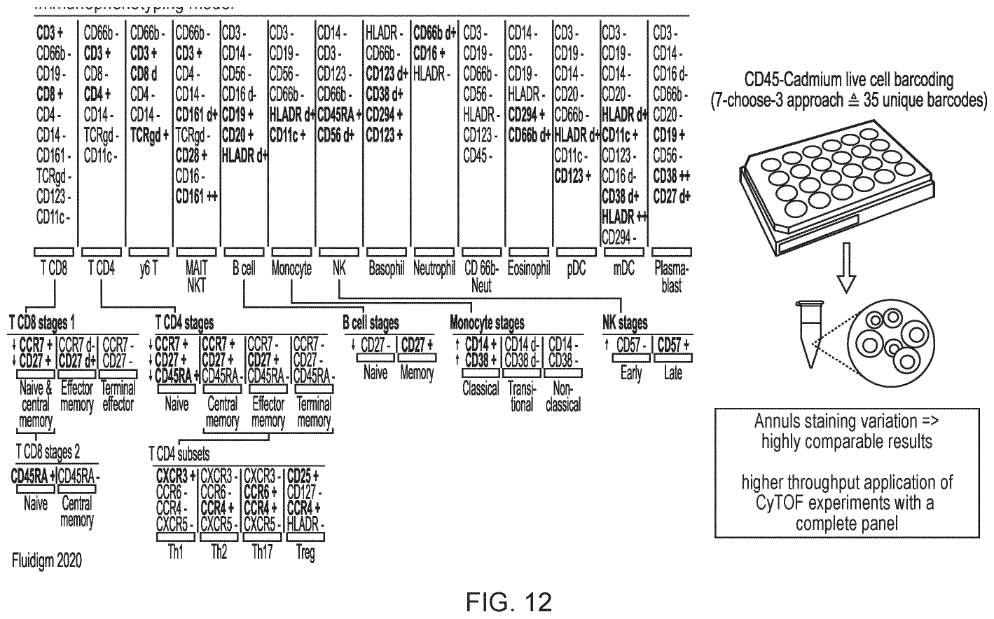

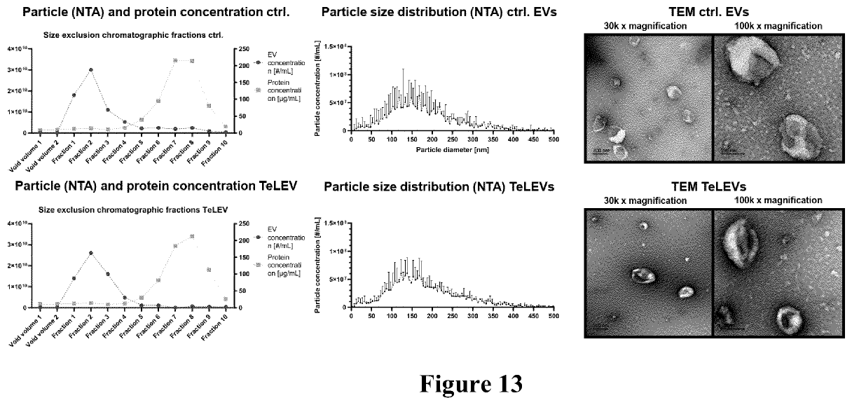

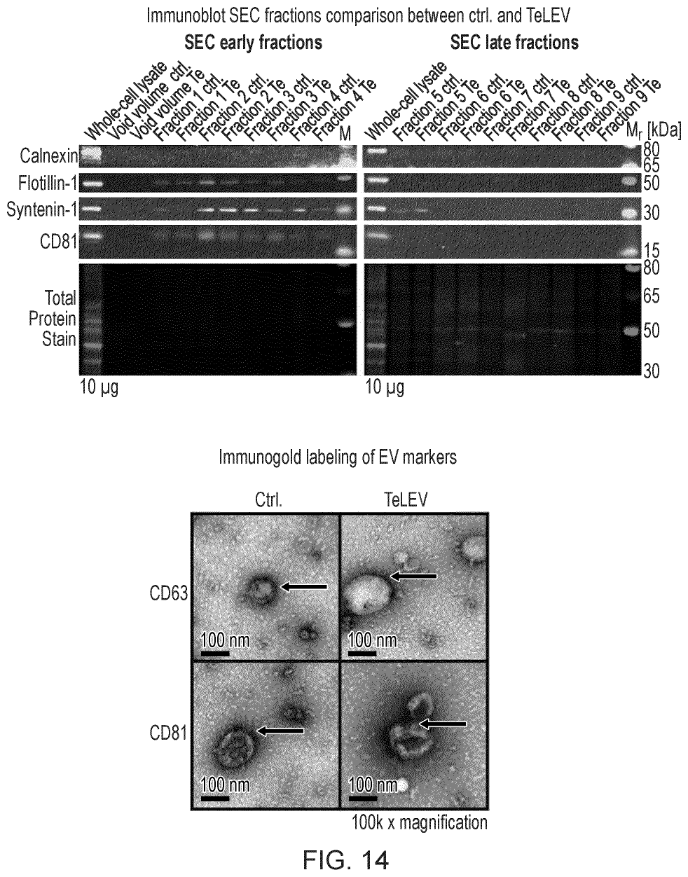

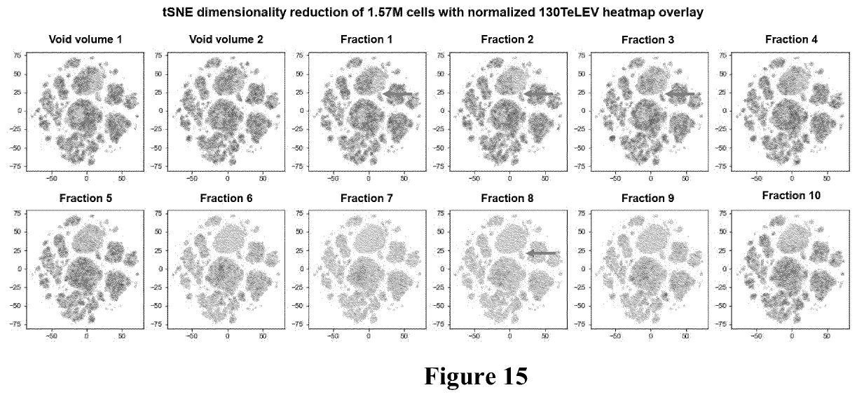

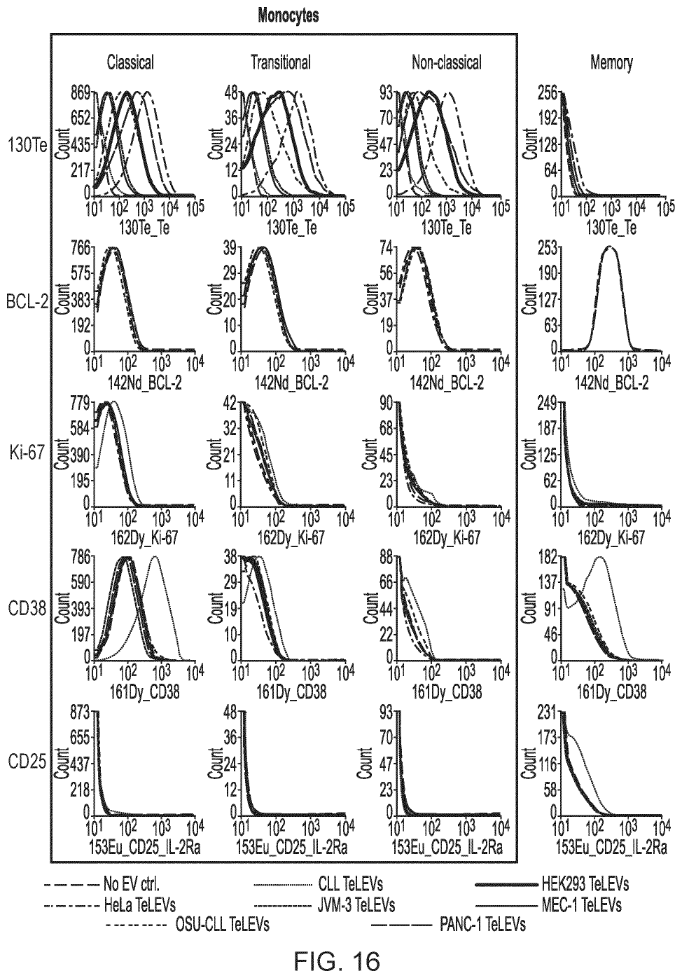

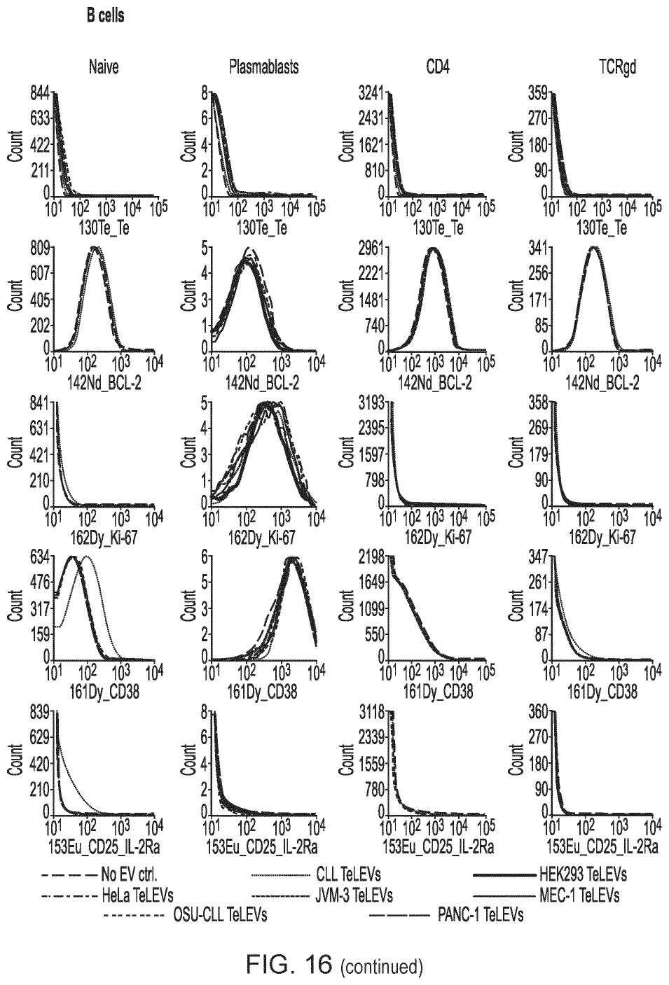

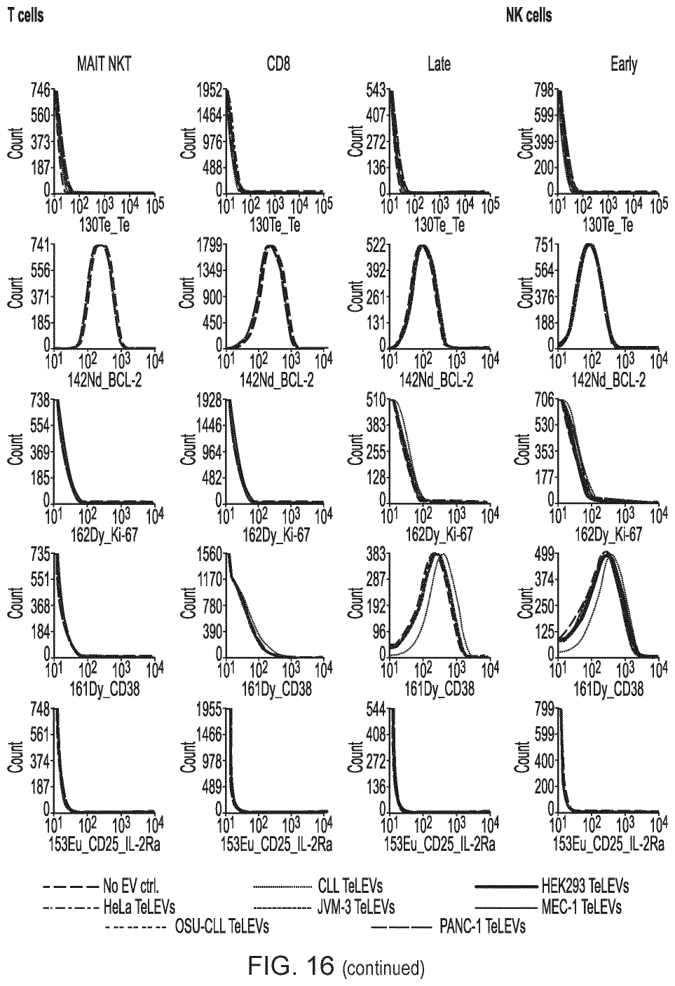

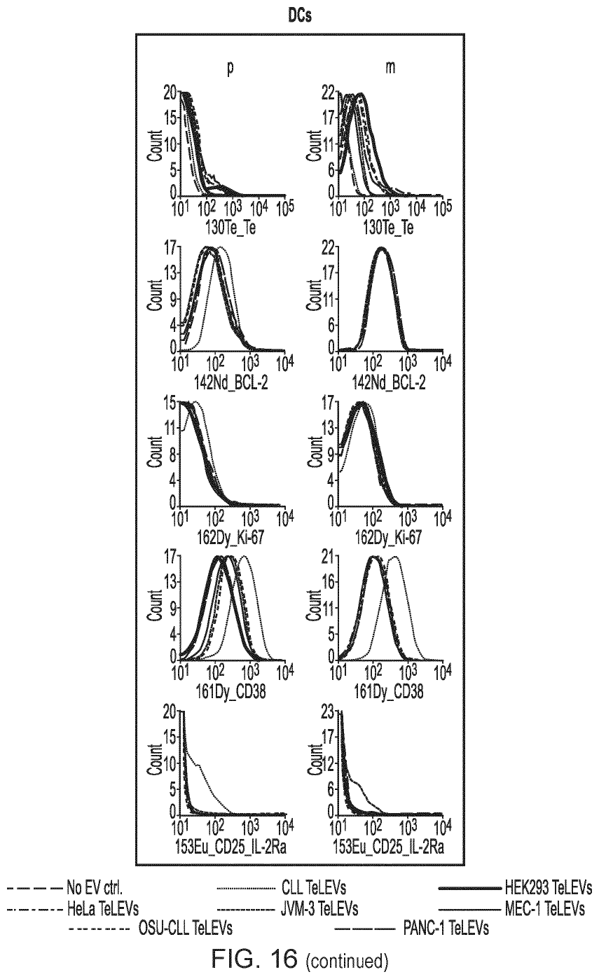

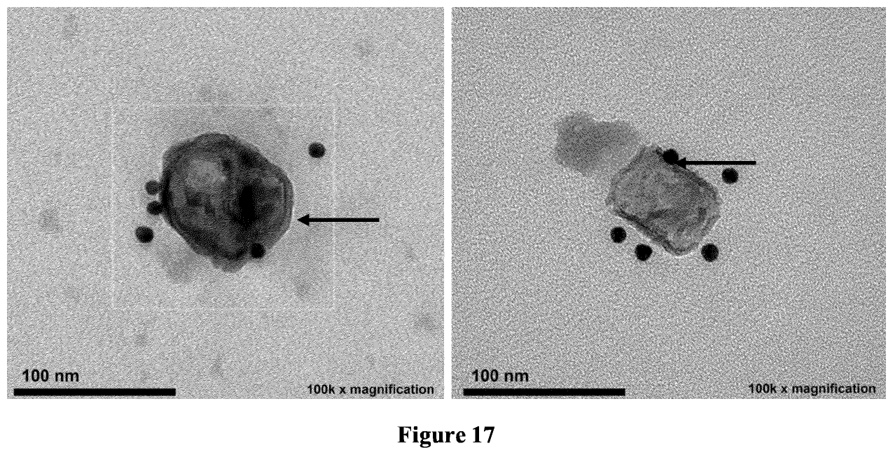

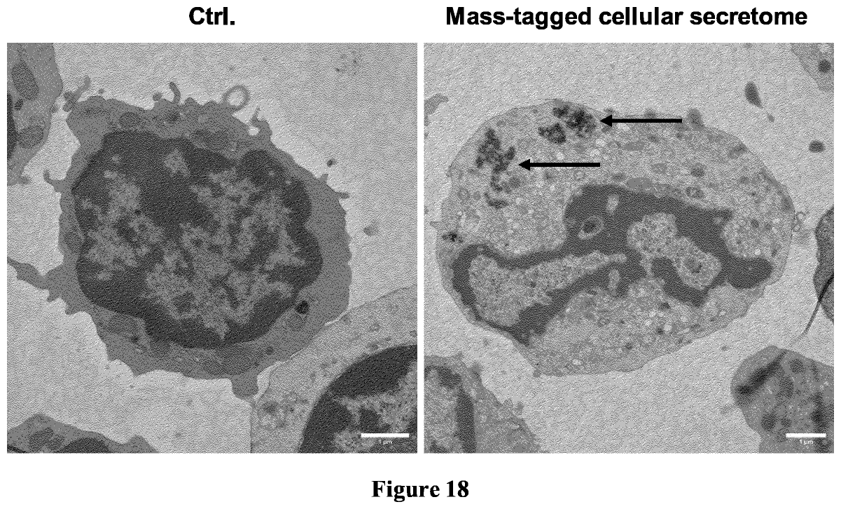

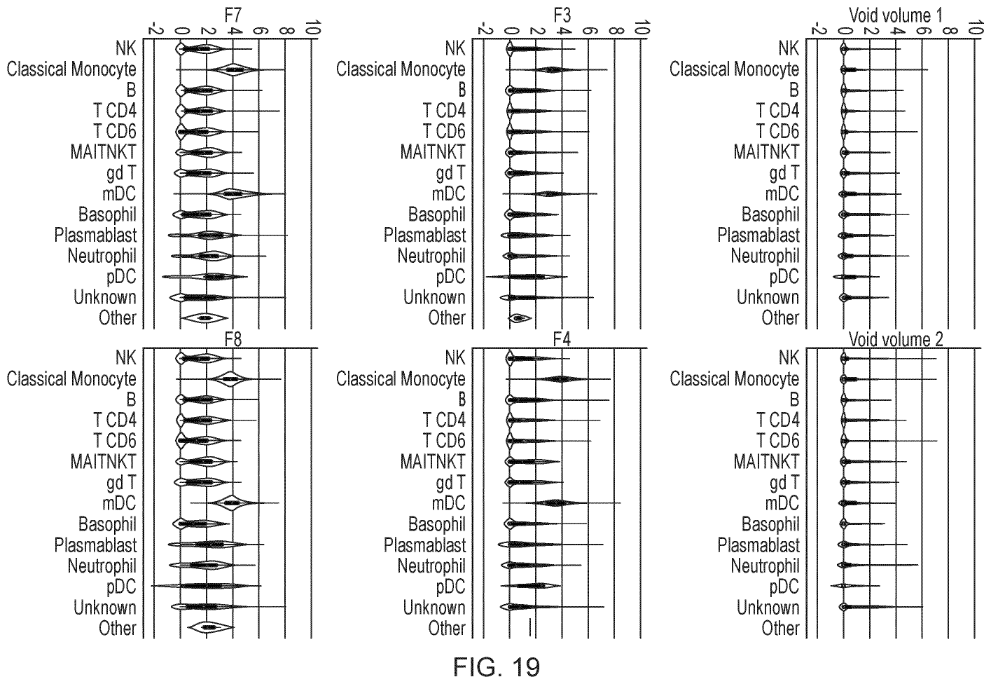

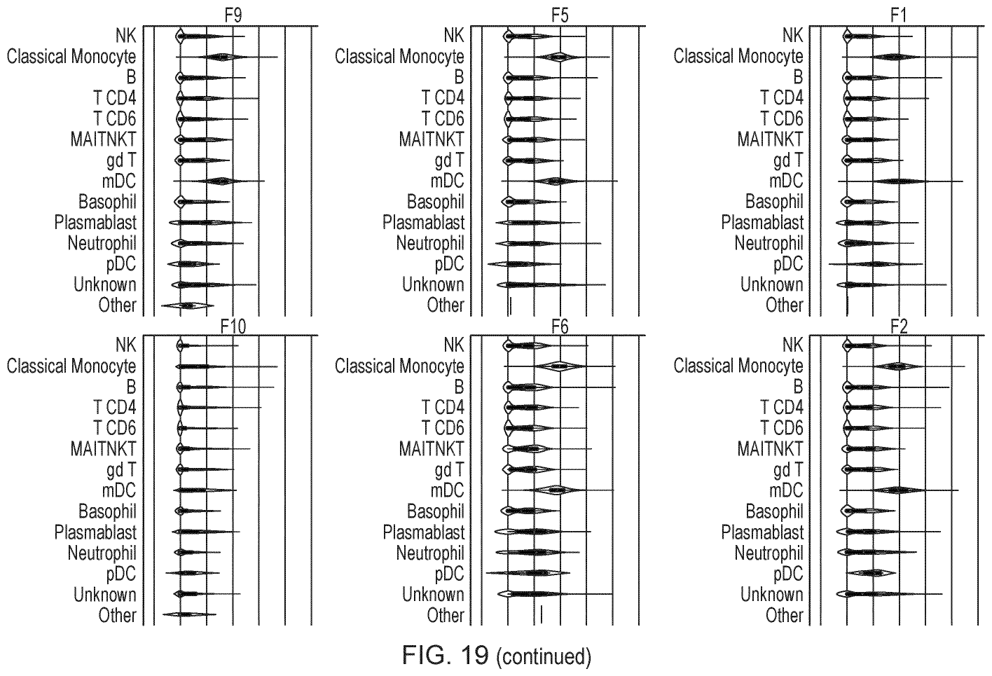

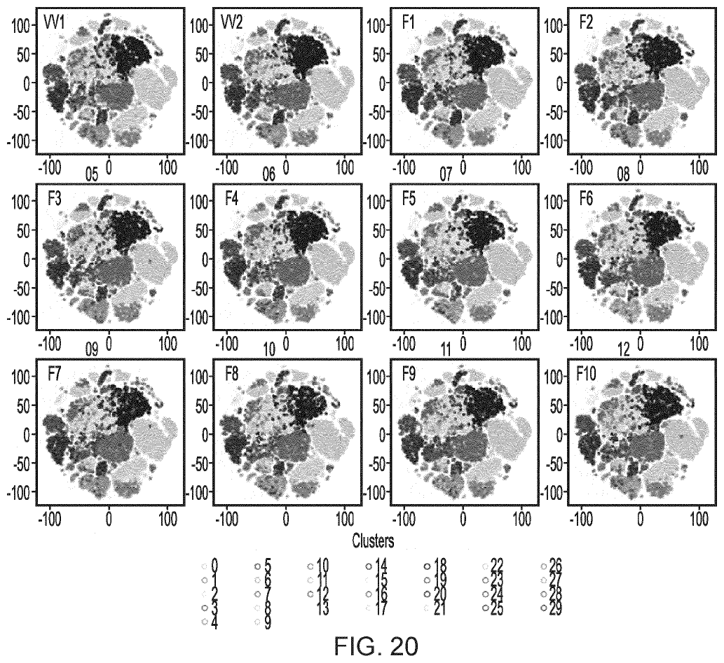

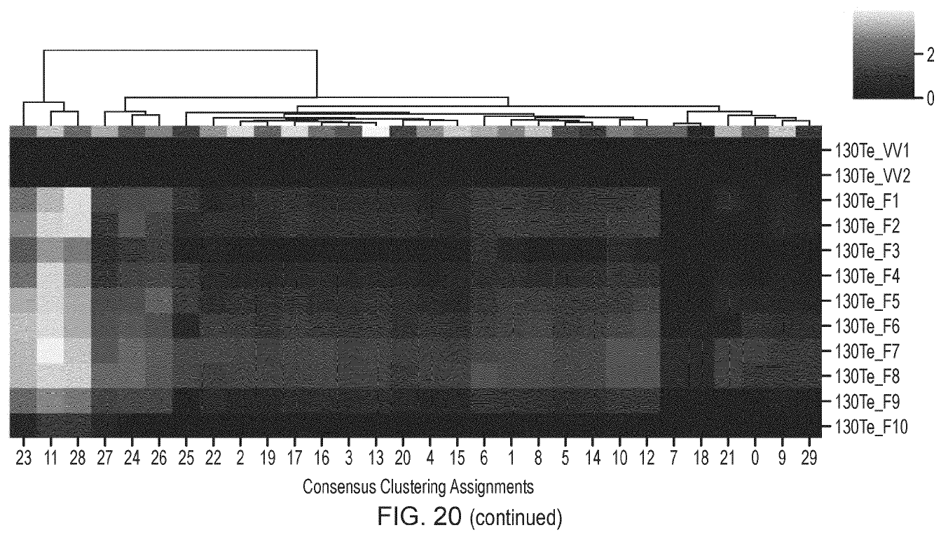

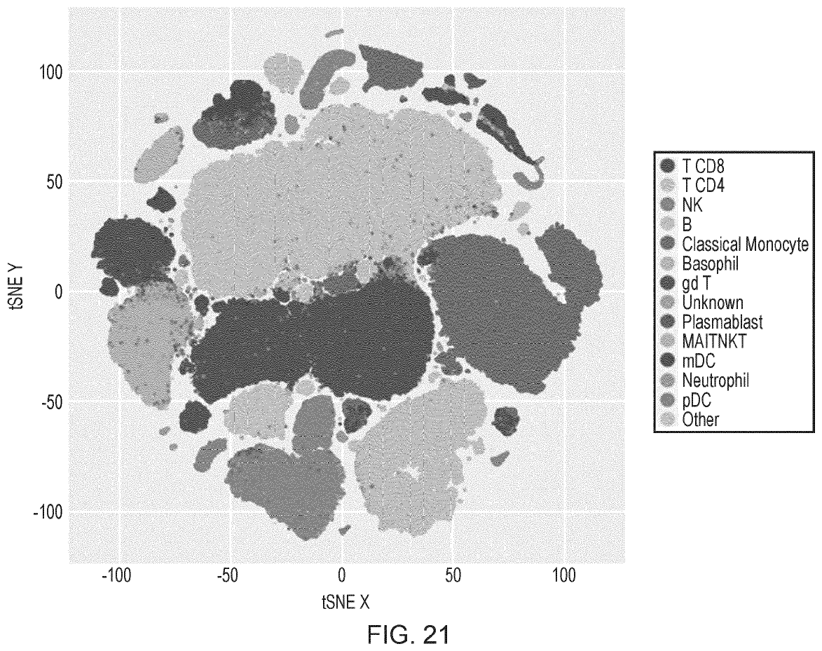

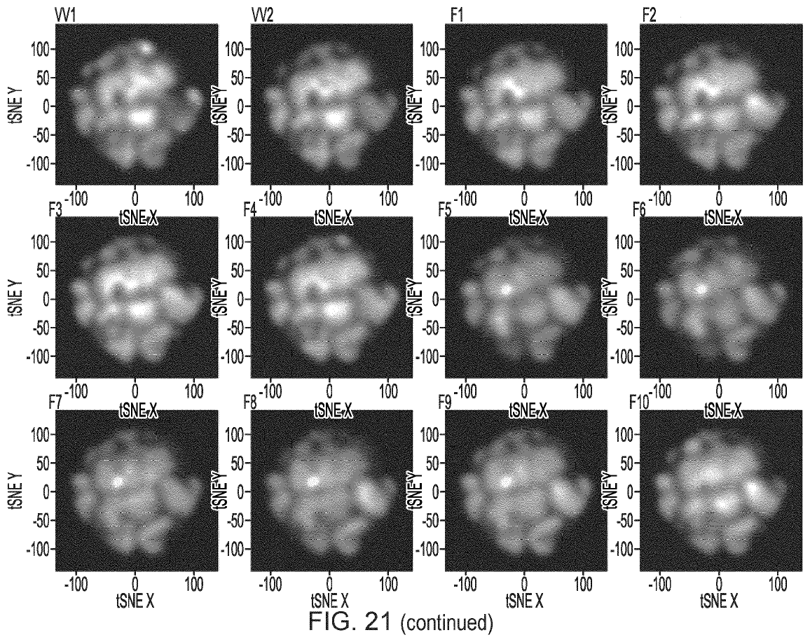

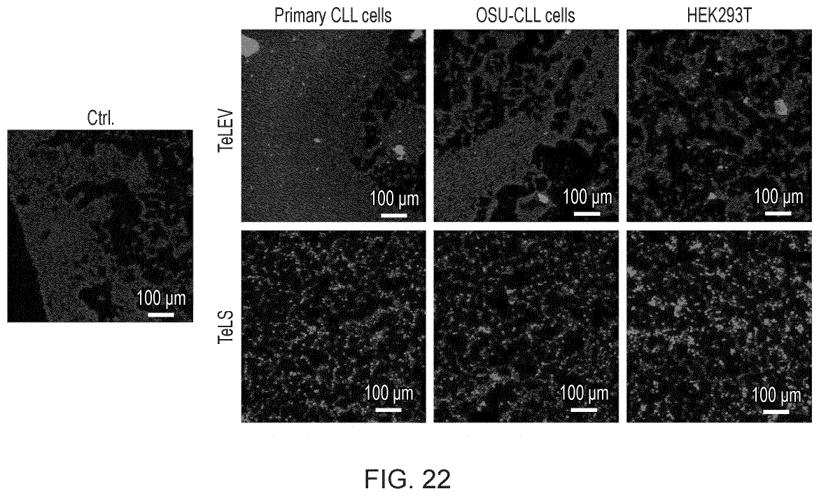

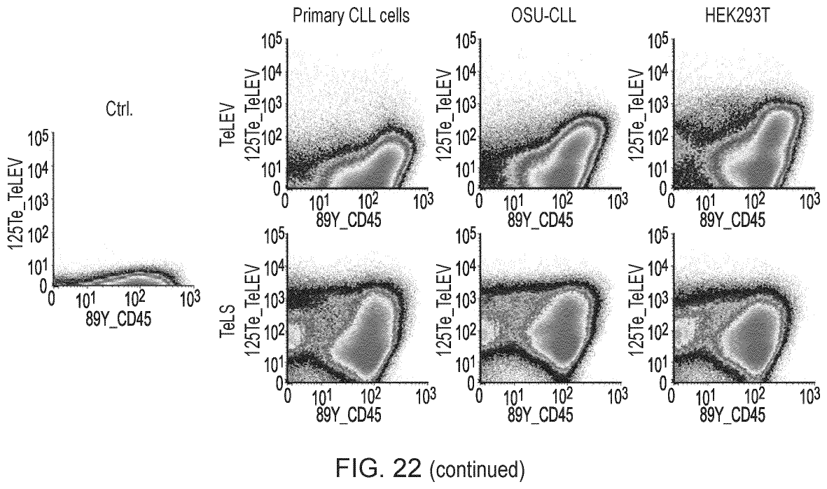

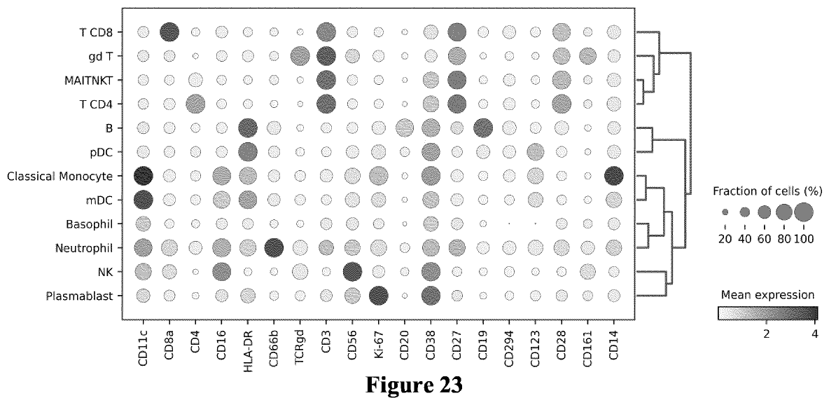

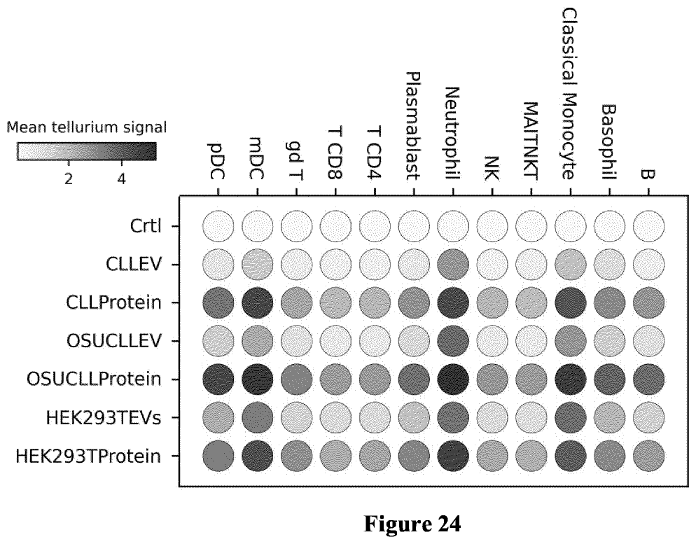

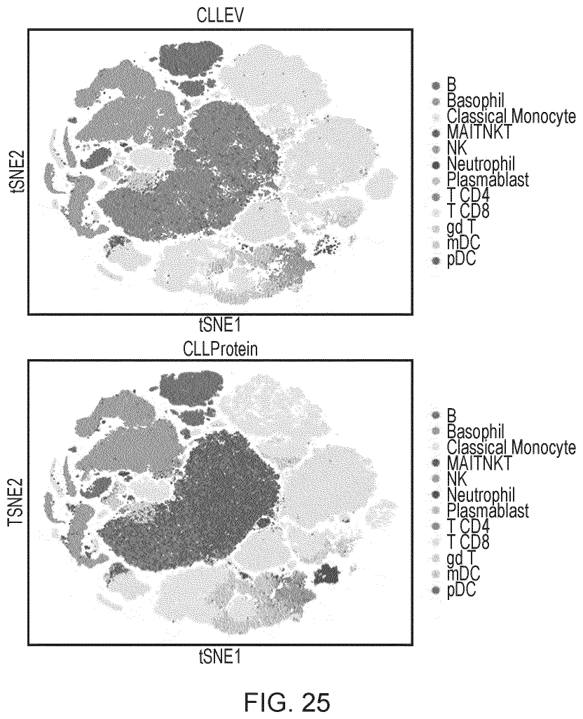

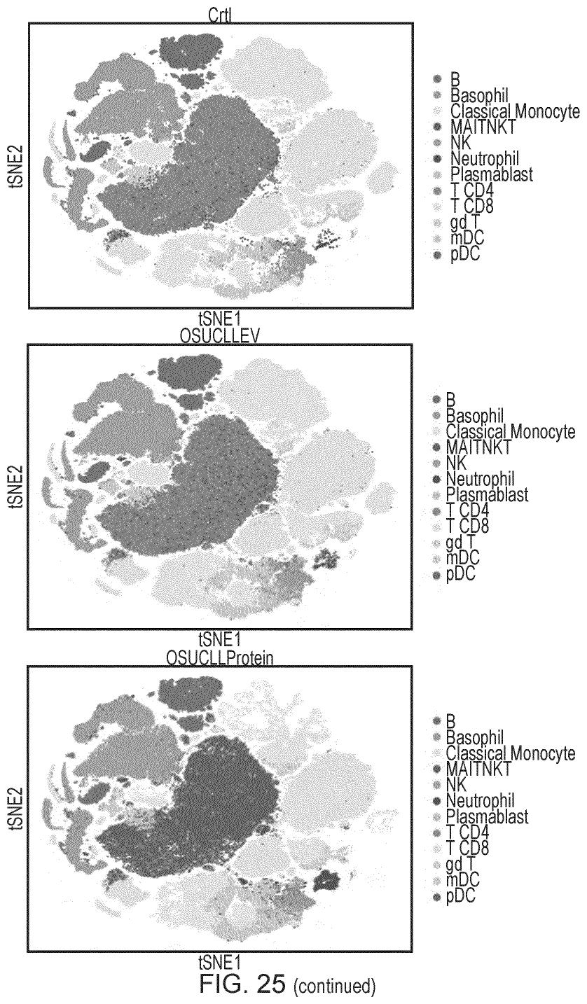

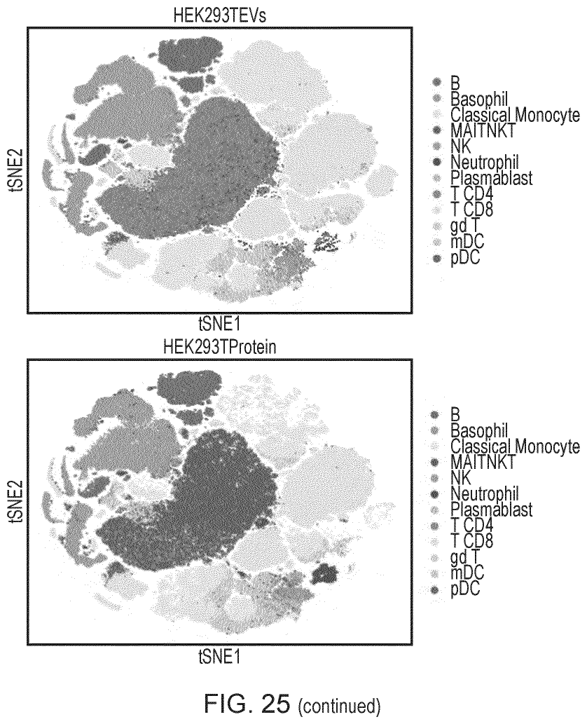

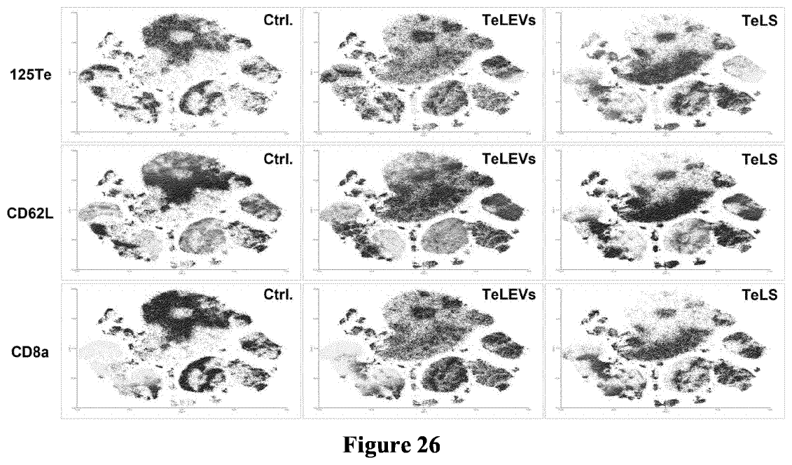

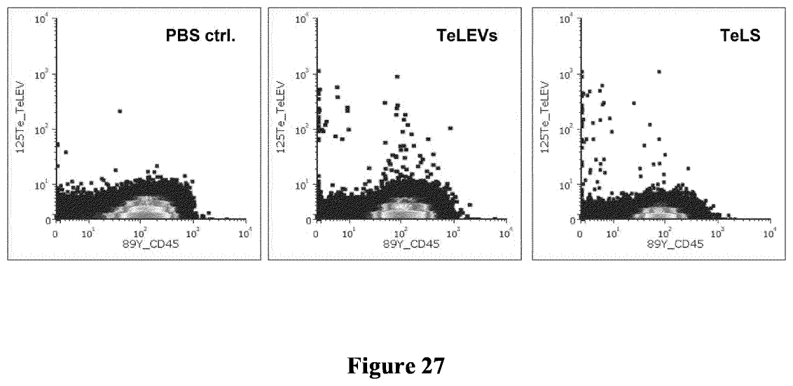

FIG. 12 shows schematically a CD45 live-cell barcoding (7-choose-3) approach the yields 35 unique barcodes that can be used to distinguish cells of different types. Variations on these, as well as other approaches, that are known or developed can be used in conjunction with the mass-tagged EVs described herein. See, for example, Muftuoglu et al. (2021) “Extended live-cell barcoding approach for multiplexed mass cytometry,” Scientific Reports 11, 12388, which describes describe a novel barcoding technique utilizing 10 different tags, seven cadmium (Cd) tags and three Pd tags, with superior signal intensities that do not impinge on lanthanide detection, which enables enhanced pooling of samples with multiple experimental conditions and markedly enhances sample throughput (which is incorporated by reference herein for this description).

Labeling can also be used to identify one or more changes in cellular function after EV uptake by using mass-tagged EVs in conjunction with other labeled components, such as antibodies, or other binding partners, for antigens/ligands associated with a change in cellular function. For example, the reagent panel shown in FIG. 11 can detect markers of DNA-damage response, chronic lymphocytic leukemia (CLL) cell biology, tyrosine kinase signaling, cell cycle, apoptosis, and checkpoints. Reagents such as these, which are also labeled with mass tags, optionally used in conjunction with mass-tagged reagents that distinguish cell type enable simultaneously detection of one or more of the following parameters: EV source or type, change in a marker of cellular function, and recipient cell source or type. A change in cellular function can be detected, for example, as a difference in the level of one or more biomarkers, or a difference in the biomarker “fingerprint” (presence or absence or relative levels of two or more biomarkers) at a time point before EV uptake and at a time point after EV uptake. Such changes can indicate a change in almost any number of cellular functions, including, but not limited to, apoptosis, DNA-damage response, migration, proliferation, and tyrosine-kinase signaling.

Mass tags can be detected by any means known in the art, including mass cytometry, mass cytometry imaging, and transmission electron microscopy, as well as other mass spectrometry-based single-cell and imaging techniques. Detection of other types of labels can be carried out by any available method suitable for the particular label.

These mass tag/labeling strategies discussed with respect to EVs apply equally to any mass-tagged soluble secretome component of interest, including viruses and proteins.

### Kits

Kits according to the invention can include one or more reagents useful for practicing one or more methods described herein. A kit generally includes a package with one or more containers holding the reagent(s) (e.g., a functionalized mass tag or mass-tagged component, such as a mass-tagged amino acid), as one or more separate compositions or, optionally, as admixture where the compatibility of the reagents will allow. The kit can also include other material(s) that may be desirable from a user standpoint, such as a buffer(s), a diluent(s), a standard(s), and/or any other material useful in sample processing, washing, or conducting any other step of the assay.

### EXAMPLE

### Introduction

For this Example, we developed the novel mass-tag labeling of the cellular secretome (e.g., thelabeling method TeLEV based on the proteomic integration of L-2-tellurienylalanine (TePhe), a tellurium-containing phenylalanine mimic, which allows for the tracking of EV uptake in heterogenous cell samples). By applying mass-tag labeling of the cellular secretome (e.g., TeLEV) with extracellular and intracellular/intranuclear mass cytometric (MC) staining with 50 markers, EV-mediated changes in cellular functions such as apoptosis, DNA-damage response, migration, proliferation, and tyrosine-kinase signaling can be analyzed in vitro and in vivo at a single-cell level and be directly correlated with EV uptake. To unite 35 samples in one tube, mass-tag labeling of the cellular secretome (e.g., TeLEV) can be combined, e.g., with a cadmium-based live-cell barcoding approach (7-choose-3), annulling staining variation and allowing for highly sensitive EV dose-response studies and functional EV uptake kinetics, inter alia.

According to MISEV2018, a broad multiplatform characterization, incl. bulk and single-EV methods, was conducted to demonstrate that Te-containing EVs were not different from unlabeled EVs. Cells from eight cell lines (including HEK293T, Hela, JVM-3) and primary cells (incl. chronic lymphocytic leukemia cells) were subjected to mass-tag labeling of the cellular secretome (e.g., TeLEV) under serum-free conditions, and sEVs were separated by the combination of 0.2 μm filtration, 10 kDa ultrafiltration, and size-exclusion chromatography (qEV/35 nm).

### Materials and Methods

### Cell Culture:

Prior to mass-tag labeling, cell lines were cultured in RPMI 1640 Medium (Gibco) supplemented with 10% FCS (Gibco), 1% GlutaMax (Gibco), and 1% Penicillin-Streptomycin (Gibco). Immediately before mass-tag labeling, primary chronic lymphocytic leukemia (CLL) cells were isolated by negative selection from EDTA-blood of CLL patients with leukocyte counts ranging from 50-100 leukocytes per nL by the addition of 50 μL RosetteSep Human B Cell Enrichment Cocktail (STEMCELL Technologies) for 20 min at RT before density gradient layering (Lymphopure, BioLegend). The viability (DAPI exclusion, DAPI-cells >95%) of cell lines and primary cells was accessed by flow cytometric analysis immediately before preparing the cell lines/primary cells for mass-tag labeling.

### Mass-Tag Labeling:

Monoisotopic L-2-tellurienylalanine (130TePhe, e.g.) diluted in water was thawed and added directly to serum-free chemically-defined medium supplemented with 1% GlutaMax (Gibco) and 1% Penicillin-Streptomycin (Gibco) in a concentration of 50 μM for all tested cell lines and primary cells. The cytotoxicity of monoisotopic TePhe was tested for all cells/cell lines to find the optimal concentration for mass-tag labeling of all components of the cellular secretome.

Cell lines or primary cells were washed twice with PBS (RT, 5 min, 350 RCF). Then, cells/cell lines were resuspended at a cell density of 1e6/mL (depending on the cell type) in TePhe-containing medium for 48 hours (depending on the cell type), yielding cell/cell line-conditioned medium (CCM) containing mass-tagged components of the cellular secretome.

### Separation/Concentration of Mass-Tagged Components of the Cellular Secretome:

First, CCM was harvested after 48 hours by centrifugation (RT, 5 min, 350 RCF). The obtained supernatant was used to purify mass-tagged extracellular vesicles and mass-tagged soluble proteins in the following steps. Next, the supernatant was centrifuged at 2000 RCF once (4° C., 10 min) and, after that, centrifuged two times at 3000 RCF (4° C., 10 min). The cleared supernatant was then subjected to 0.2 μm filtration depending on the component of the cellular secretome remaining to be separated/concentrated.

50 mL of filtered CCM was centrifuged (4° C., 20 min, 3000 RCF) in a 10 kDa MWCO ultrafiltration unit (Vivaspin Turbo 15 RC, Sartorius), yielding 500 μL of CCM concentrate. Next, this 500 μL was layered on top of a size-exclusion chromatography column (qEVoriginal/35 nm, Izon), and EV purification was performed according to the manufactures instructions. To recover the majority of mass-tagged EVs with high-purify, 2.5 mL of void volume was discarded, and 400 μL fractions were collected. The first five EV-containing fractions (2 mL) were pooled and concentrated to 200 μL by 10 kDa MWCO ultrafiltration. All other eluting fractions were as well collected in 400 μL fractions containing mass-tagged soluble proteins of the cellular secretome (such as antibodies, cytokines, and hormones). After concentration/separation, mass-tagged samples were ready to be used in mass cytometric and imaging mass cytometric assays.

### Transmission Electron Microscopy (TEM) Imaging of EVs:

5 μl of diluted sample was loaded on top of copper grids (formvar-coated, Science Services) and left incubating for 20 minutes, as described previously [6]. After being fixed with 2% paraformaldehyde for 5 min, samples were washed with PBS and fixed again with 1% glutaraldehyde for 5 min. The grids were washed with Milli-Q water and contrasted (or left uncontrasted) for 4 min with or without 1.5% uranyl acetate. EVs were imaged using a Gatan OneView 4K camera mounted on a Jem-2100Plus (Jeol) operating at 200 kV.

### Mass Cytometric Analysis of EV Recipient Cells

1e6 of freshly purified peripheral blood mononuclear cells in 1 mL supplemented RPMI 1640 medium were cultured for 16 h in the presence of mass-tagged EVs from different cells/cell lines. EVs were dosed according to cell equivalents of 2e6 cells conditioning media for 48 hours. Next, cells were washed, live-cell barcoded (anti-CD45-Cd conjugates), pooled, and counted. 10e6 barcoded cells were used for mass cytometric staining. According to the manufacturer's instructions, cells were sequentially stained with the Maxpar Direct Immune Profiling Assay (MDIPA, Fluidigm) and other surface and intracellular/intranuclear markers before being freshly fixed (1.6% FA in PBS) and incubated overnight with 125 nM Cell-ID Intercalator-Ir (Fluidigm). At least 1e6 cells at a concentration of 5e5 cells per mL in Cell Acquisition Solution (Fluidigm) with 0.1×EQ Four Element Calibration Calibration Beads (Fluidigm) were acquired with a Helios mass cytometer (Fluidigm). Data were normalized in the CyTOF software (Fluidigm), debarcoded (ParkerICI/premessa, FR package), and cleaned up and analyzed using FCS Express 7 (De Novo software). Opt-SNE and FlowSOM algorithms were used for dimensionality reduction and clustering. Analyzed data were visualized as tSNE plots and multi-dimensional heatmaps to evaluate EV uptake and phenotypical and functional changes in EV recipient cells.

### CONCLUSION

In conclusion, the present invention provides a novel mass-tag labeling approach for studying the interactions and effects of cellular secretome components, including extracellular vesicles (EVs), soluble proteins, and viruses, on recipient cells' phenotype and function. This approach overcomes the limitations of existing labeling methods, such as reduced efficiency, high cost, and incompatibility with high-dimensional single-cell analysis. The mass-tag labeling approach described herein ensures uniform and normalized labeling of various secretome components, eliminates the need for electroporation or other modifications, and is compatible with mass cytometry, imaging mass cytometry, electron microscopy-based techniques, and other mass spectrometry-based single-cell and imaging techniques. This novel method offers improved labeling efficiency, enhanced multiplexing capability, simplified workflow, reduced cost, adaptability, and increased sensitivity and specificity, making it a versatile and powerful tool for researchers studying cellular secretomes.

### REFERENCES

[1] van Niel, G., D'Angelo, G., & Raposo, G. (2018). Shedding light on the cell biology of extracellular vesicles. Nature Reviews. Molecular Cell Biology, 19(4), 213-228.
[2]Möller, A., & Lobb, R. J. (2020). The evolving translational potential of small extracellular vesicles in cancer. Nature Reviews. Cancer, 20(12), 697-709.
[3] Herrmann, I. K., Wood, M. J. A., & Fuhrmann, G. (2021). Extracellular vesicles as a next-generation drug delivery platform. Nature Nanotechnology, 16(7), 748-759.
[4] Colombo, M., Raposo, G., & Théry, C. (2014). Biogenesis, secretion, and intercellular interactions of exosomes and other extracellular vesicles. Annual Review of Cell and Developmental Biology, 30, 255-289.
[5] van Niel, G., Carter, D. R. F., Clayton, A., Lambert, D. W., Raposo, G., & Vader, P. (2022). Challenges and directions in studying cell-cell communication by extracellular vesicles. Nature Reviews. Molecular Cell Biology, 1-14.
[6] Bachurski, D., Schuldner, M., Nguyen, P.-H., Malz, A., Reiners, K. S., Grenzi, P. C., Babatz, F., Schauss, A. C., Hansen, H. P., Hallek, M., & Pogge von Strandmann, E. (2019). Extracellular vesicle measurements with nanoparticle tracking analysis—An accuracy and repeatability comparison between NanoSight NS300 and ZetaView. Journal of Extracellular Vesicles, 8(1), 1596016.
[7] Verweij, F. J., Balaj, L., Boulanger, C. M., Carter, D. R. F., Compeer, E. B., D'angelo, G., El Andaloussi, S., Goetz, J. G., Gross, J. C., Hyenne, V., & Others. (2021). The power of imaging to understand extracellular vesicle biology in vivo. Nature Methods, 18(9), 1013-1026.
[8] Ramirez, M. I., Amorim, M. G., Gadelha, C., Milic, I., Welsh, J. A., Freitas, V. M., Nawaz, M., Akbar, N., Couch, Y., Makin, L., Cooke, F., Vettore, A. L., Batista, P. X., Freezor, R., Pezuk, J. A., Rosa-Fernandes, L., Carreira, A. C. O., Devitt, A., Jacobs, L., . . . Dias-Neto, E. (2018). Technical challenges of working with extracellular vesicles. Nanoscale, 10(3), 881-906.
[9] Simonsen, J. B. (2019). Pitfalls associated with lipophilic fluorophore staining of extracellular vesicles for uptake studies. Journal of Extracellular Vesicles, 8(1), 1582237.
[10] Théry, C., Witwer, K. W., Aikawa, E., Alcaraz, M. J., Anderson, J. D., Andriantsitohaina, R., Antoniou, A., Arab, T., Archer, F., Atkin-Smith, G. K., & Others. (2018). Minimal information for studies of extracellular vesicles 2018 (MISEV2018): a position statement of the International Society for Extracellular Vesicles and update of the MISEV2014 guidelines. Journal of Extracellular Vesicles, 7(1), 1535750.
[11] Adapted from “Extracellular Vesicle Separation by Density Gradient” by BioRender.com (2022). Retrieved from https://app.biorender.com/biorender-templates.
[12]S. H. Ranganath, O. Levy, M. S. Inamdar, and J. M. Karp, “Harnessing the Mesenchymal Stem Cell Secretome for the Treatment of Cardiovascular Disease,” Cell Stem Cell, vol. 10, no. 3, 2012, doi: 10.1016/j.stem.2012.02.005.
[13]C. R. Harrell, C. Fellabaum, N. Jovicic, V. Djonov, N. Arsenijevic, and V. Volarevic, “Molecular Mechanisms Responsible for Therapeutic Potential of Mesenchymal Stem Cell-Derived Secretome,” Cells, vol. 8, no. 5, 2019, doi: 10.3390/cells8050467.
[14]C. Giesen et al., “Highly multiplexed imaging of tumor tissues with subcellular resolution by mass cytometry,” Nat. Methods, vol. 11, no. 4, 2014, doi: 10.1038/nmeth.2869.
[15]M. Le Rochais, P. Hemon, J.-O. Pers, and A. Uguen, “Application of High-Throughput Imaging Mass Cytometry Hyperion in Cancer Research,” Front. Immunol., vol. 13, 2022, Accessed: May 2, 2023. [Online]. Available: https://www.frontiersin.org/articles/10.3389/fimmu.2022.859414
[16]J. Bassan et al., “TePhe, a tellurium-containing phenylalanine mimic, allows monitoring of protein synthesis in vivo with mass cytometry,” Proc. Natl. Acad. Sci. U.S.A, vol. 116, no. 17, pp. 8155-8160, April 2019, doi: 10.1073/pnas.1821151116.
[17]J. Wang et al., “Loading of metal isotope-containing intercalators for mass cytometry-based high-throughput quantitation of exosome uptake at the single-cell level,” Biomaterials, vol. 255, p. 120152, October 2020, doi: 10.1016/j.biomaterials.2020.120152.
[18]J. Wang et al., “High-throughput single-cell analysis of exosome mediated dual drug delivery, in vivo fate and synergistic tumor therapy,” Nanoscale, vol. 12, no. 25, pp. 13742-13756, July 2020, doi: 10.1039/d0nr02344b.
[19]H. Liu, Y. Tian, C. Xue, Q. Niu, C. Chen, and X. Yan, “Analysis of extracellular vesicle DNA at the single-vesicle level by nano-flow cytometry,” J. Extracell. Vesicles, vol. 11, no. 4, p. e12206, 2022, doi: 10.1002/jev2.12206.
[20]K. B. Johnsen et al., “Evaluation of electroporation-induced adverse effects on adipose-derived stem cell exosomes,” Cytotechnology, vol. 68, no. 5, pp. 2125-2138, October 2016, doi: 10.1007/s10616-016-9952-7.
[21]J. Shin et al., “Comparative analysis of differentially secreted proteins in serum-free and serum-containing media by using BONCAT and pulsed SILAC,” Sci. Rep., vol. 9, no. 1, 2019, doi: 10.1038/s41598-019-39650-z.

What is claimed is:

1. An extracellular vesicle (EV), wherein a component of the EV is labeled with at least one mass tag.

2. A plurality of EVs according to claim 1.

3. The plurality of EVs of claim 2, wherein the plurality comprises EVs from more than one sample.

4. The plurality of EVs of claim 3, wherein the EVs from each different sample are distinguished by a different detectable label or combination of detectable labels.

5. The plurality of EVs of claim 4, wherein the different labels or combinations of labels comprise different mass tags or combinations of mass tags.

6. A method of producing a mass-tagged soluble component from a production cell, the method comprising:
exposing at least one production cell to a mass-tagged component that can be taken up by the production cell; and
purifying a mass-tagged soluble component produced by the production cell.

7. The method of claim 6, wherein the mass-tagged soluble component is selected from an extracellular vesicle (EV), a virus particle, a cellular secretome, an EV proteome or secretome, or a component of any of the foregoing.

8. The method of claim 7, wherein the mass-tagged component is a mass-tagged EV.

9. The method of any one of claims 6-8, wherein the production cell is exposed to the mass-tagged component under serum-free conditions.

10. The method of any one of claims 6-9, wherein the production cell is derived from a cell line, optionally selected from HEK293T, HeLa, OSU-CLL, and PANC-1.

11. The method of claim 7 or claim 9, wherein the production cell is derived from a primary cell, optionally a chronic lymphocytic leukemia cell.

12. The method of any one of claims 7-11, wherein the EVs are purified by a method comprising filtration, ultrafiltration, and size-exclusion chromatography.

13. The method of claim 12, wherein the filtration comprises 0.2 μM filtration, the ultrafiltration comprises 10 kDa ultrafiltration, and the size-exclusion chromatograph comprises qEV/35 nm chromatography.

14. A method of producing a mass-tagged EV, the method comprising contacting the EV with a mass tag that is functionalized to bind to a component of the EV under conditions suitable for that binding to occur.

15. The method of claim 14, wherein the method additionally comprises purifying the EV from a bodily fluid or tissue before contacting the EV with the functionalized mass tag.

16. An EV produced according to the method of any one of claims 7-14.

17. An extracellular vesicle proteome or secretome from the EV of claim 16, wherein the proteome or secretome comprises a mass-tagged component.

18. A method of using the EV of claim 1, the method comprising:
contacting the EV with a recipient cell, whereby the recipient cell takes up the EV.

19. The method of claim 18, wherein the method is an in vivo method, and the EV is used for diagnosis or therapy.

20. The method of claim 18, wherein the EV is used in a non-diagnostic and non-therapeutic method.

21. The method of claim 18, wherein the method is an in vitro method.

22. The method of claim 18, wherein the method comprises a biodistribution study.

23. The method of claim 19, wherein the method comprises analyzing a single recipient cell.

24. The method of claim 19, wherein the method comprises analyzing a plurality of recipient cells.

25. The method of claim 24, wherein the plurality of recipient cells comprises cells of different cell types.

26. The method of claim 18, wherein the method additionally comprises measuring a change in cellular function after EV uptake, as compared to before EV uptake, wherein the change in cellular function is optionally selected from apoptosis, DNA-damage response, migration, proliferation, and tyrosine-kinase signaling.

27. The method of any one of claims 18-26, wherein the recipient cell is labeled with at least one detectable label.

28. The method of claim 27, wherein the detectable label indicates a characteristic of the recipient cell.

29. The method of claim 28, wherein the characteristic of the recipient cell, alone or in combination with other characteristics, distinguishes the recipient cell type from at least one other cell type.

30. The method of claim 29, wherein the characteristic of the recipient cell, alone or in combination with other characteristics, identifies the recipient cell type.

31. The method of any one of claims 27-30, wherein the detectable label comprises a mass tag.

32. The method of any one of claims 27-30, wherein the recipient cell is subjected to CD45-based live cell barcoding or palladium-based fixed cell barcoding.

33. The method of claim 32, where the barcoding identifies cells from different samples and/or cells of different cell types.

34. The method of any one of claims 18-33, wherein the method comprises employing the detectably labeled recipient cell and/or one or more detectably labeled reagents to characterize EV uptake and/or EV-mediated effects, to identify recipient cells, and/or in a multiplex analysis, optionally wherein the one or more detectably labeled reagents are one or more antibodies.

35. The method of claim 34, wherein the detectably labeled recipient cells are labeled using a metal-labeled antibody panel and/or the one or more detectably labeled reagents comprise a metal-labeled antibody panel.

36. The method of any one of claims 18-31, wherein the method comprises performing a technique selected from mass cytometry, mass cytometry imaging, and transmission electron microscopy on the recipient cell.

37. A recipient cell produced by the method of claim 18.

38. A method of detecting the EV of claim 1 or claim 16 and or the recipient cell of claim 37, wherein the method comprises performing a technique selected from mass cytometry, mass cytometry imaging, and transmission electron microscopy.

39. A kit for performing the method of claim 6, wherein the kit comprises one or more mass-tagged components that can be taken up by a production cell.

40. The EV of claim 1 or claim 16, the plurality of EVs of any one of claims 2-5, the method of any one of claims 7-13, 18-36, or 38, the recipient cell of claim 37, or the kit of claim 39, wherein said mass-tagged component comprises an amino acid or analog thereof.

41. The EV, method, or kit of claim 40, wherein the amino acid is phenylalanine or an analog thereof.

42. The EV, method, or kit of claim 40 or claim 41, wherein a protein component of the EV, virus particle, or cellular or EV secretomeis labeled with the at least one mass tag.

43. The EV of claim 1 or claim 16, the plurality of EVs of any one of claims 2-5, the method of any one of claims 7-13, 18-36, or 38, the recipient cell of claim 37, the EV or method of any one of claims 40-42, or the kit of claim 39, wherein the mass tag comprises an organotellurophene tag.

44. The EV, method, or kit of claim 43, wherein the organotellurophene tag comprises L-2-tellurienylalanine (TePhe) or TeMal.

45. The EV, method, or kit of claim 44, wherein a plurality of mass tags selected from isotopologues of TePhe or TeMal is provided or employed to facilitate multiplex analysis.

46. The EV or method of claim 43 or claim 44, wherein the mass-tagged EV does not differ substantially from an unlabeled EV produced from the same cell type under the same conditions as the labeled EV.

47. The EV or method of claim 46, wherein the mass-tagged EV and the unlabeled EV have substantially the same effect(s) on a recipient cell.

48. The EV or method of claim 47, wherein the effect(s) of the mass-tagged EV and the unlabeled EV differ by no more than ±15, ±14, ±13±12, ±11, ±10, ±9, ±8, ±7, ±6, ±5, ±4, ±3, ±2, ±1, ±0.5% percent.

49. The EV or method of claim 46, wherein the mass-tagged EV and the unlabeled EV have substantially the same MISEV2018 characteristic(s) for one or more or all MISEV2018 characteristics.

50. The EV or method of claim 47, wherein the characteristic(s) of the mass-tagged EV and the unlabeled EV differ by no more than ±15, ±14, ±13±12, ±11, ±10, ±9, ±8, ±7, ±6, ±5, ±4, ±3, ±2, ±1, ±0.5% percent.

51. The use of a mass tag, characterized in that the mass tag is used to label the cellular secretome, and a mass-tagged component of the cellular secretome is purified.

52. The use of claim 51, characterized in that the cellular secretome is labeled by metabolic labeling.

53. The use of claim 51 or claim 52, characterized in that the mass-tagged component of the cellular secretome comprises one or a plurality of EV(s).

54. The use of any one of claims 51-53, characterized in that the mass-tagged component of the cellular component is used in a study with one or a plurality of other detectably labeled component(s).

55. The use of claim 54, characterized in that the study comprises a multiplex analysis.

56. An EV according to claim 1, for use in an in vivo method of diagnosis or therapy, the method comprising contacting the EV with a recipient cell, whereby the recipient cell takes up the EV.
